(19) United States
(12) Patent Application Publication
(10) Pub. No.: US 2023/0031995 A1
(43) Pub. Date: Feb. 2, 2023

(54) Motion-Based Cardiopulmonary Function Index Measuring Device, and Senescence Degree Prediction Apparatus and Method

(71) Applicant: DYPHI INC., Daejeon (KR)

(72) Inventors: Seong Jun Yoon, Seongnam-si (KR); Hyun Chul Roh, Daejeon (KR); Hee Won Jung, Bucheon-si (KR)

(21) Appl. No.: 17/757,971

(22) PCT Filed: Oct. 20, 2020
(86) PCT No.: PCT/KR2020/014342

§ 371 Date: Jun. 24, 2022

(30) Foreign Application Priority Data
Dec. 26, 2019 (KR) 10-2019-0175504
May 26, 2020 (KR) 10-2020-0063186
Oct. 20, 2020 (KR) 10-2020-0135721

(51) Int. Cl. G16H 50/30; A61B 5/0205; A61B 5/083; A61B 5/11; A61B 5/22; G16H 20/30; G16H 50/20

(52) U.S. Cl. CPC G16H 50/30; A61B 5/0205; A61B 5/083; A61B 5/11; A61B 5/22; G16H 20/30; G16H 50/20

(57) ABSTRACT

Provided is a method of predicting a cardiopulmonary function comprising measuring motion of a cardiopulmonary function measurement-target person, by a motion sensor, determining a gait speed of the cardiopulmonary function measurement-target person according to the motion of the cardiopulmonary function measurement-target person, by a processor, and predicting a cardiopulmonary function index of the cardiopulmonary function measurement-target person, based on the gait speed of the cardiopulmonary function measurement-target person, by the processor.

### TECHNICAL FIELD

The present disclosure relates to a motion-based cardiopulmonary function index measurement device and a frailty index prediction device and method, and, more particularly, to a device and method for predicting a cardiopulmonary function index and a frailty index based on motion information of a subject.

### BACKGROUND ART

Measurement of a cardiopulmonary function is very important, for risk evaluation and treatment response evaluation of cardiovascular diseases such as congestive heart failure or pulmonary hypertension, respiratory diseases such as chronic obstructive pulmonary disease or interstitial lung disease and oncological diseases such as lung cancer requiring pneumonectomy. However, in cardiopulmonary exercise test, etc., since a subject needs to exercise up to a maximum intensity according to instructions of a test protocol, it is difficult for the elderly or patients having poor physical functions to perform a complicated cardiopulmonary function test. In addition, since medical equipments and experts for the cardiopulmonary function test are available only in some general hospitals, the cardiopulmonary function test may be restrictively performed in general surgery. In addition, there is a difficulty in periodically repeating time-consuming and costly professional cardiopulmonary function test for a short period of time for the purpose of a follow-up test of treatment response.

However, a cardiopulmonary function measurement method capable of repeatedly and conveniently performing measurement may be helpful for accurate and appropriate clinical decision making. Accordingly, there is a need for a better method capable of replacing the existing professional cardiopulmonary function test in a normal patient and easily measuring a cardiopulmonary function.

Meanwhile, in most clinical sites, the physical functions of a subject is evaluated based on an eye measurement method using a classic tool such as a stopwatch or a tape measure. The eye measurement method is relatively inexpensive and easy to learn, but is capable of restrictively obtaining information, is labor-intensive and has a large variation in test results.

In order to solve these problems, a body function evaluation method using a gait analyzer and a marker-based motion capture device has been proposed. However, body function evaluation requires a complex functional test for the entire body, such as subject's gait, balance and muscle strength, but existing devices may still obtain only fragmentary information. For example, the gait analyzer may obtain only gait information of the subject. In addition, the motion capture device may obtain motion information of the entire body of the subject. However, a measurement environment is limited (a laboratory equipped with a plurality of infrared cameras is essential), a marker needs to be attached to a subject's body part (which causes inconvenience and interference with natural movement of the subject), a separate experimental environment needs to be set before and after measurement, a separate data analysis process is required, and thus real-time operation is not possible. In addition, it is difficult to use the motion capture device in a general clinical environment.

Therefore, there is a need for an automated physical function evaluation device and method capable of overcoming limitations in manual measurement and enabling precise and quantitative analysis.

### DISCLOSURE

### Technical Problem

In this specification, provided is a method and apparatus for predicting a cardiopulmonary function index based on motion information of a cardiopulmonary function measurement-target person. In addition, provided is a method and apparatus for estimating motion information of a subject using a single distance sensor and a single image sensor and predicting a frailty index of the subject. In addition, a system for predicting a cardiopulmonary function index or frailty index of a subject is provided. In addition, a program for executing a method of predicting a cardiopulmonary function index or frailty index of a subject and a storage medium storing the program are provided.

### Technical Solution

A method of predicting a cardiopulmonary function according to embodiments of the present disclosure may comprise measuring motion of a cardiopulmonary function measurement-target person, by a motion sensor, determining a gait speed of the cardiopulmonary function measurement-target person according to the motion of the cardiopulmonary function measurement-target person, by a processor, and predicting a cardiopulmonary function index of the cardiopulmonary function measurement-target person, based on the gait speed of the cardiopulmonary function measurement-target person, by the processor.

A device for predicting a cardiopulmonary function according to embodiments of the present disclosure may comprise a motion sensor configured to measure motion of a cardiopulmonary function measurement-target person, a memory configured to store a program including at least one instruction, and a processor configured to predict a cardiopulmonary function index of the cardiopulmonary function measurement-target person, by executing the at least one program. The at least one instruction may comprise an instruction for enabling the motion sensor to measure motion of the cardiopulmonary function measurement-target person, an instruction for enabling the processor to determine at least one of a gait speed or gait acceleration of the cardiopulmonary function measurement-target person according to the motion of the cardiopulmonary function measurement-target person, and an instruction for enabling the processor to predict a cardiopulmonary function index of the cardiopulmonary function measurement-target person, based on the gait speed or gait acceleration of the cardiopulmonary function measurement-target person.

A motion-based frailty index prediction method according to embodiments of the present disclosure is performed by a frailty index prediction device comprising a sensor and a processor. The motion-based frailty index prediction method may comprise obtaining distance information and image information of a subject, by the sensor, estimating motion information of the subject using the distance information and the image information, by the processor, extracting a physical function parameter of the subject from the motion information, by the processor, and predicting a frailty index of the subject based on the physical function parameter, by the processor. The sensor may comprise a single distance sensor for obtaining the distance information and a single image sensor for obtaining the image information.

A computer program product according to embodiments of the present disclosure may comprise instructions performed by a computer for a motion-based frailty index prediction method. The motion-based frailty index prediction method may be performed by a frailty index prediction device comprising a sensor and a processor, and the motion-based frailty index prediction method may comprise obtaining distance information and image information of a subject, by the sensor, estimating motion information of the subject using the distance information and the image information, by the processor, extracting a physical function parameter of the subject from the motion information, by the processor, and predicting a frailty index of the subject based on the physical function parameter, by the processor, and the sensor may comprise a single distance sensor for obtaining the distance information and a single image sensor for obtaining the image information.

A motion-based frailty index prediction device according to embodiments of the present disclosure may comprise a single distance sensor configured to detect distance information of a subject, a single image sensor configured to detect image information of the subject, and a processor configured to predict a frailty index of the subject, the processor may estimate motion information of the subject using the distance information and the image information, extract a physical function parameter of the subject from the motion information, and predict the frailty index of the subject based on the extracted physical function parameter.

### Advantageous Effects

According to a cardiopulmonary function prediction method provided in the present disclosure, the cardiopulmonary function of a subject may be predicted simply and efficiently. In addition, according to a frailty index prediction method provided in the present disclosure, a frailty index of a subject maybe predicted simply and efficiently.

### DESCRIPTION OF DRAWINGS

FIG. 1 shows an embodiment of a gait measurement device for determining a cardiopulmonary function index based on a gait ability of a cardiopulmonary function measurement-target person.

FIG. 2 shows an embodiment of a cardiopulmonary function index measurement system including a gait measurement device, an oxygen saturation measurement device, an external computing device and a server.

FIG. 3 shows an embodiment of a cardiopulmonary function index prediction method performed by the gait measurement device.

FIG. 4 shows an embodiment of a cardiopulmonary function index prediction method according to a cardiopulmonary function measurement system including the gait measurement device 210 and the external computing device.

FIG. 5 shows an embodiment of a cardiopulmonary function index prediction method according to the cardiopulmonary function measurement system including the gait measurement device, the external computing device and the server.

FIG. 6 shows an embodiment of a cardiopulmonary function index prediction method according to the cardiopulmonary function measurement system including the gait measurement device, the oxygen saturation measurement device, the external computing device and the server.

FIG. 7 shows an embodiment of a setting item of a gait speed measurement item shown on a display, in a program performed in the gait measurement device or the external computing device.

FIG. 8 shows an embodiment of a gait speed measurement item of the gait speed measurement item shown in a display, in a program performed in the gait measurement device or the external computing device.

FIG. 9 shows an embodiment of a data management item shown on a display, in a program performed by the gait measurement device or the external computing device.

FIG. 10 shows a method of measuring a gait speed according to an embodiment.

FIG. 11 shows a graph showing a gait distance of each group according to each New York Heart Association (NYHA) functional classification according to a 6-minute gait test.

FIG. 12 shows graphs illustrating correlation between a gait distance according to a 6-minute gait test and a cardiac output (CO), TPR (Total Pulmonary Resistance), Peak VO2 (peak exercise oxygen consumption), AT (anaerobic threshold), Oxygen Pulse and VE-VCO2 slope (regression slope relating minute ventilation to carbon dioxide output).

FIG. 13 shows a graph of a Kaplan-Meier survival curve of two groups according to a 6-minute gait test. The Kaplan-Meier survival curve represents a survival rate of an experimental group over time.

FIG. 14 is a view illustrating a frailty index prediction device according to an embodiment of the present disclosure.

FIG. 15 is a view illustrating a configuration of a processor in a frailty index prediction device according to an embodiment of the present disclosure.

FIG. 16 shows a specific embodiment of extracting physical function parameters.

FIGS. 17a and 17b are two-dimensional graphs respectively illustrating a relationship between chronological age and gait speed and a relationship between a frailty index and a gait speed.

FIG. 18 is a graph illustrating the survival rates of groups having certain gait speeds by Kaplan-Meier analysis.

FIG. 19 illustrates distributions of the gait speed of older adults living in a community in Korea according to the genders.

FIG. 20 is a graph for estimating a frailty index by using a gait speed and other measured values.

FIG. 21 shows frailty index prediction sensitivity and specificity by a physical function parameter.

FIG. 22 is a flowchart illustrating a frailty index prediction method according to an embodiment of the present disclosure.

In the following description, the same reference numerals are used for the same elements, and overlapping descriptions are omitted, unless described in other drawings.

### MODE FOR INVENTION

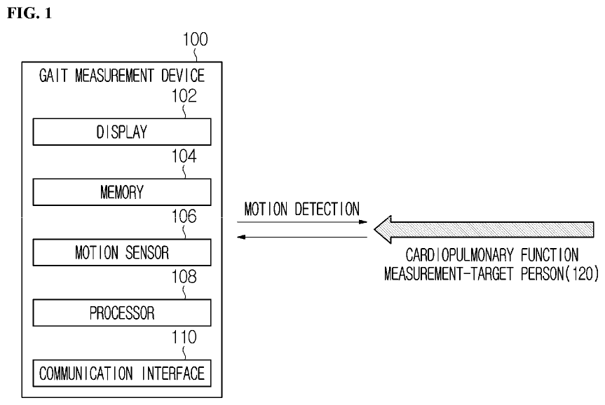

FIG. 1 shows an embodiment of a gait measurement device 100 for determining a cardiopulmonary function index based on a gait ability of a cardiopulmonary function measurement-target person 120.

The gait measurement device 100 may include a display 102, a memory 104, a motion sensor 106, a processor 108 and a communication interface 110. In some embodiments, the gait measurement device 100 may further include components necessary for measurement of the gait ability of the cardiopulmonary function measurement-target person 120.

The gait measurement device 100 may include the display 102 for displaying data related to measurement of the gait ability of the cardiopulmonary function measurement-target person 120 and a predicted cardiopulmonary function. For example, the display 102 may display a value indicating the gait ability of the cardiopulmonary function measurement-target person 120, such as step length, step width, stride length, number of steps, gait speed and gait acceleration of the cardiopulmonary function measurement-target person 120. The step length represents a distance from one heel to the other heel. In addition, the stride length represents a distance from one heel to the same heel after two steps. In addition, the display 102 may display data related to the cardiopulmonary function for the cardiopulmonary function measurement-target person 120. The data related to the cardiopulmonary function may include cardiopulmonary function indices such as mean Pulmonary Arterial Pressure (mPAP,), Cardiac Output (CO), Total Pulmonary Resistance (TPR), New York Heart Association (NYHA) Classification, peak exercise oxygen consumption (Peak VO2), anaerobic threshold (AT), Oxygen Pulse, and regression slope relating minute ventilation to carbon dioxide output (VE-VCO2 slope).

In addition, the display 102 may include a touchscreen function for receiving a user's instruction. Accordingly, a user may receive information through the display 102 and input a user's instruction to be performed by the gait measurement device 100. For example, the gait measurement device 100 may receive, from the user, personal information of the cardiopulmonary function measurement-target person 120, such as identification information, age, gender, residential area, race and the like through the display 102. Alternatively, the gait measurement device 100 may receive setting information for measurement of the gait ability from the display 102.

The gait measurement device 100 may include an input interface other than the display 102. Accordingly, the gait measurement device 100 may receive information for cardiopulmonary function prediction from the other input interface such as a mechanical or electronic button in addition to the display 102.

The gait measurement device 100 may include the memory 104 storing a program for driving the gait measurement device 100. The memory 104 may store at least one program for performing cardiopulmonary function prediction. In addition, the memory 104 may store information obtained from the communication interface 110 or store information processed in the processor 108. In addition, the memory 104 may store information on correlation between the cardiopulmonary function index and the gait ability, necessary for information processing of the processor 108.

The gait measurement device 100 may include the motion sensor 106. By the motion sensor 106, how the cardiopulmonary function measurement-target person 120 moved during a measurement time is detected. Sound waves or infrared rays are emitted from the motion sensor 106, and the sound waves or infrared rays reflected from the cardiopulmonary function measurement-target person 120 are detected by the motion sensor 106, thereby identifying the position of the cardiopulmonary function measurement-target person 120. The motion sensor 106 may be implemented as a one-dimensional sensor capable of measuring only a distance from a measured object or a two-dimensional sensor capable of measuring the position of the measured object, but is not limited to the above embodiment. A person skilled in the art may use a commonly known distance measuring sensor as the motion sensor 106. When the motion sensor 106 is a two-dimensional sensor capable of measuring the position of the cardiopulmonary function measurement-target person 120 on a flat surface, vertical and horizontal components of the position of the cardiopulmonary function measurement-target person 120 may be detected. In addition, According to the vertical and horizontal components of the position of the cardiopulmonary function measurement-target person 120, the vertical and horizontal components of the gait speed and gait acceleration of the cardiopulmonary function measurement-target person 120 may be calculated. In addition, according to the vertical and horizontal components of the gait speed and gait acceleration of the cardiopulmonary function measurement-target person 120, the cardiopulmonary function index of the cardiopulmonary function measurement-target person 120 may be predicted.

The gait measurement device 100 may include the processor 108. The processor 108 may perform an instruction of at least one program for performing measurement of the gait ability stored in the memory 104 and prediction of cardiopulmonary function index. Hereinafter, the function of the processor 108 for performing measurement of the gait ability and prediction of the cardiopulmonary function index will be described.

By the processor 108, based on the motion information of the cardiopulmonary function measurement-target person 120, elements regarding the gait ability of the cardiopulmonary function measurement-target person 120, such as the step length, step width, stride length, number of steps, gait distance, gait speed, gait acceleration and the like of the cardiopulmonary function measurement-target person 120, may be calculated. In addition, the processor 108 may calculate elements regarding the gait ability of the cardiopulmonary function measurement-target person 120 based on at least one of setting values. For example, the setting information includes at least one of a measurement direction, a measurement range or an effective measurement range.

The measurement direction represents the motion direction of the cardiopulmonary function measurement-target person to be recognized by the motion sensor 106. The measurement direction may include a forward direction, a backward direction, a left direction and a right direction, etc. based on the motion sensor 106. The motion sensor 106 may record motion of the cardiopulmonary function measurement-target person 120 when the cardiopulmonary function measurement-target person 120 moves in a set measurement direction.

The measurement range represents a range of a distance between the motion sensor 106 capable of measuring motion of cardiopulmonary function measurement-target person and the cardiopulmonary function measurement-target person 120. The motion sensor 106 may generate motion information of the cardiopulmonary function measurement-target person 120, when detecting that the cardiopulmonary function measurement-target person 120 is in the measurement range. The user may input the measurement range to the gait measurement device 100. Alternatively, the measurement range may automatically vary according to the measurement environment of the motion sensor 106.

The effective measurement range represents a range of an area in which the motion sensor 106 detects the cardiopulmonary function measurement-target person 120, in order to generate the motion information of the cardiopulmonary function measurement-target person 120. Even when the cardiopulmonary function measurement-target person 120 walks within the measurement range, if walking is not completed within the effective measurement range, the processor 108 does not generate the motion information of the cardiopulmonary function measurement-target person 120. Similarly to the measurement range, the user may input the effective measurement range to the gait measurement device 100. Alternatively, the effective measurement range may automatically vary according to the measurement environment of the motion sensor 106.

The processor 108 may generate motion information according to one of a gait speed measurement mode in which the gait speed of the cardiopulmonary function measurement-target person is measured and a gait analysis mode in which motion information according to gait analysis of the cardiopulmonary function measurement-target person is additionally obtained in addition to the gait speed.

The processor 108 may predict the cardiopulmonary function index of the cardiopulmonary function measurement-target person 120, based on the motion information of the cardiopulmonary function measurement-target person 120. The motion information such as the gait speed and the gait acceleration is closely related to the cardiopulmonary function of the cardiopulmonary function measurement-target person 120. Accordingly, the processor 108 may calculate the cardiopulmonary function index of the cardiopulmonary function measurement-target person 120 according to correlation between the motion information and the cardiopulmonary function index.

The processor 108 may determine a gait speed parameter indicating the gait speed. The gait speed parameter represents the gait speed of a specific section. For example, the gait speed parameter may be defined according to the section having a size of 0.2 m/s. As a specific example, the gait speed parameter may be defined as 1 for a section of 0.4 to 0.6 m/s and may be defined as 2 for a section of 0.6 to 0.8 m/s. In addition, for the remaining sections of 0.2 m/s, a unique gait speed parameter may be defined. Similarly, the processor 108 may determine a gait acceleration parameter representing the gait acceleration of a specific section. The above example is merely an example, and the value of the gait speed parameter and the corresponding section thereof and the value of the gait acceleration parameter and the corresponding section thereof may be easily changed by a person skilled in the art.

The processor 108 may determine the cardiopulmonary function index from the gait speed, by correlation of the gait speed-cardiopulmonary function index indicating the relationship between the gait speed and the cardiopulmonary function index. When the gait speed is expressed as the gait speed parameter, the processor 108 may determine the cardiopulmonary function index according to the correlation of the gait speed-cardiopulmonary function index, from the gait speed parameter. The correlation of the gait speed-cardiopulmonary function index may be determined according to regression analysis or machine learning of statistical data on the gait speed and the cardiopulmonary function index. The correlation of the gait speed-cardiopulmonary function index will be described in detail with reference to FIGS. 11 to 13.

Additionally, the processor 108 may calculate the gait acceleration of the cardiopulmonary function measurement-target person 120 from the motion information of the cardiopulmonary function measurement-target person 120. In addition, the processor 108 may obtain the cardiopulmonary function index of the cardiopulmonary function measurement-target person 120, by analyzing the gait speed and the gait acceleration of the cardiopulmonary function measurement-target person 120.

The processor 108 may derive the cardiopulmonary function index of the cardiopulmonary function measurement-target person 120, by further considering the personal information such as identification information, actual age, gender, residential area, race, height, weight, etc. of the cardiopulmonary function measurement-target person 120. The processor 108 may predict the probability of occurrence of a cardiopulmonary disease according to the cardiopulmonary function index. Accordingly, according to the probability of occurrence of the cardiopulmonary disease calculated by the processor 108, it may be helpful in determining a treatment method of the cardiopulmonary function measurement-target person 120.

The processor 108 may determine the cardiopulmonary function index using not only motion information but also other measured values. In order to measure the cardiopulmonary function index, other factors may be additionally used, in addition to the motion information such as the gait speed. For example, data obtained through muscle strength, muscle mass evaluation and balance sense evaluation may be additionally considered. In order to obtain the additional data, separate measurement devices may be installed separately from the gait measurement device 100. For example, a sthenometer may be installed for muscle strength evaluation, a muscle mass meter may be installed for muscle mass evaluation, and a balance sense measurement device may be installed for balance sense evaluation.

The processor 108 may perform calculation for deriving the cardiopulmonary function index based on statistical data. For example, the statistical data related to the correlation of the gait speed-cardiopulmonary function index, the gait speed, and statistical data according to the actual age and gender of the cardiopulmonary function measurement-target person 120 may be used. In addition, statistical data on other factors associated with the cardiopulmonary function index may be used. The processor 108 may periodically update the statistical data from an external server and store it in the memory 104 of the gait measurement device 100.

The processor 108 may obtain the cardiopulmonary function index of the cardiopulmonary function measurement-target person 120 calculated in a server 122 through the communication interface 110, without calculating the cardiopulmonary function index of the cardiopulmonary function measurement-target person 120. When the cardiopulmonary function index of the cardiopulmonary function measurement-target person 120 is predicted in the server 122, the size of data and programs necessary for prediction of the cardiopulmonary function index stored in the memory 104 of the gait measurement device 100 may be reduced, and leakage of the motion information and the personal information necessary for prediction of the cardiopulmonary function index may be limited.

The processor 108 may obtain identification information of a user. In addition, when the identification information of the user is valid, the processor 108 may perform a function for determining the cardiopulmonary function index. The processor 108 may transmit the identification information of the user stored in the external server through the communication interface 110 to determine whether the identification information of the user is valid. In addition, upon determining that the identification information of the user is valid by the external server, the processor 108 may allow user access to a function for cardiopulmonary function index prediction. In addition, the processor 108 may store information related to the cardiopulmonary function measurement-target person 120 in a user's email account.

The processor 108 may generate at least one time-based graph between the gait speed and the gait acceleration of the cardiopulmonary function measurement-target person 120. In addition, the time-based graph may be displayed on the display 102. The processor 108 may predict the cardiopulmonary function index of the cardiopulmonary function measurement-target person 120 according to a gait pattern shown in the time-based graph.

The processor 108 may predict the cardiopulmonary function index of the cardiopulmonary function measurement-target person 120, by further considering the oxygen saturation of the cardiopulmonary function measurement-target person 120. Oxygen saturation refers to a ratio of the number of hemoglobin bonded to oxygen to the total number of hemoglobin in the blood. The oxygen saturation may be measured from the cardiopulmonary function measurement-target person 120 before and/or after motion information measurement.

The gait measurement device 100 may include an additional processor for performing an instruction necessary for cardiopulmonary function prediction, in addition to the processor 108. In addition, the gait measurement device 100 may perform an instruction related to prediction and determination of the cardiopulmonary function index using the processor outside the gait measurement device 100.

The gait measurement device 100 may include the communication interface 110 to receive and transmit data from and to an external device. The communication interface 110 may be implemented according to the communication standards such as mobile communication, Bluetooth, Wi-Fi, infrared data association standard (IrDA), WiMAX, etc.

In addition, the gait measurement device 100 may transmit the motion information and personal information of the cardiopulmonary function measurement-target person 120 to the external device through the communication interface 110. In addition, the gait measurement device 100 may obtain the cardiopulmonary function index of the cardiopulmonary function measurement-target person 120 and medical information based on the cardiopulmonary function index from the external device through the communication interface 110. In addition, in some embodiments, the gait measurement device 100 may obtain a database indicating a relationship between the cardiopulmonary function index and the gait speed from the external device through the communication interface 110.

The gait measurement device 100 of FIG. 1 is not limited to the above-described embodiment, and those of ordinary skill in the field of medical devices for cardiopulmonary diseases may easily exclude or modify some components of the gait measurement device 100 described with reference to FIG. 1 or add components well-known to those skilled in the art to the gait measurement device 100.

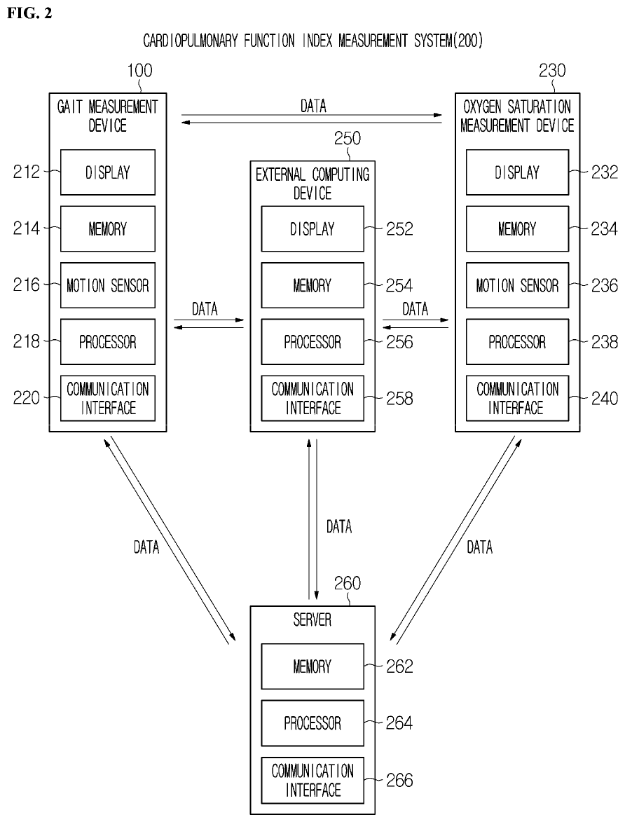

FIG. 2 shows an embodiment of a cardiopulmonary function index measurement system 200 including a gait measurement device 210, an oxygen saturation measurement device 230, an external computing device 250 and a server 260.

The cardiopulmonary function index measurement system 200 may include at least one of the oxygen saturation measurement device 230, the external computing device 250 or the server 260, together with the gait measurement device 210. The gait measurement device 210 may include a display 212, a memory 214, a motion sensor 216, a processor 218 and a communication interface 220. The gait measurement device 210 may be communicatively connected to the oxygen saturation measurement device 230, the external computing device 250 and the server 260 to transmit and receive data. In addition, the gait measurement device 210 of FIG. 2 may perform the function of the gait measurement device 100 of FIG. 1. Accordingly, the relationship among the gait measurement device 100 described in FIG. 1, the external server and the external device becomes clearer by cardiopulmonary function index measurement system 200 shown in FIG. 2.

The oxygen saturation measurement device 230 may include a display 232, a memory 234, an oxygen saturation sensor 236, a processor 238 and the communication interface 240.

The display 232 may display the oxygen saturation of the cardiopulmonary function measurement-target person 120. In addition, the display 232 may display data related to the cardiopulmonary function for the cardiopulmonary function measurement-target person 120.

The memory 234 may store at least one program for performing oxygen saturation measurement. In addition, the memory 234 may store information obtained from the communication interface 240 or information processed in the processor 238. In addition, the memory 234 may store information on correlation between the cardiopulmonary function index and oxygen saturation, necessary for information processing of the processor 238.

According to the oxygen saturation sensor 236, the oxygen saturation of the cardiopulmonary function measurement-target person 120 may be measured before and/after motion information measurement.

The processor 238 may perform at least one instruction for performing measurement of oxygen saturation stored in the memory 234 and/or prediction of the cardiopulmonary function index. In addition, the processor 238 of the oxygen saturation measurement device 230 may perform the function of the processor 108 of the gait measurement device 100 instead. Accordingly, by the processor 238 of the oxygen saturation measurement device 230, the cardiopulmonary function index may be predicted from the gait ability and the oxygen saturation.

In some embodiments, the oxygen saturation measurement device 230 may include an additional processor for performing an instruction necessary for cardiopulmonary function prediction, in addition to the processor 238. In addition, the oxygen saturation measurement device 230 may perform an instruction for prediction and determination of the cardiopulmonary function index using the processor outside the oxygen saturation measurement device 230.

The communication interface 240 may be implemented according to the communication standard such as mobile communication, Bluetooth, Wi-Fi, infrared data association (IrDA), WiMAX and the like. Through the communication interface 240, the oxygen saturation information and personal information of the cardiopulmonary function measurement-target person 120 may be transmitted to the gait measurement device 210, the external computing device 250, and/or the server 260. In addition, through the communication interface 110, the cardiopulmonary function index of the cardiopulmonary function measurement-target person 120 and the medical information based on the cardiopulmonary function index may be obtained from the gait measurement device 210, the external computing device 250, and/or the server 260. In addition, in some embodiments, the oxygen saturation measurement device 230 may obtain a cardiopulmonary function prediction database indicating a relationship between the cardiopulmonary function index and the oxygen saturation from the gait measurement device 210, the external computing device 250, and/or the server 260 through the communication interface 240.

The external computing device 250 may include a display 252, a memory 254, a processor 256, and a communication interface 258. The external computing device 250 may be a personal computer, a laptop, a mobile device or a wearable device.

The external computing device 250 may be communicatively connected to the gait measurement device 210 and/or the oxygen saturation measurement device 230 to obtain motion information, personal information and/or oxygen saturation information of the cardiopulmonary function measurement-target person 120 from the gait measurement device 210 and/or the oxygen saturation measurement device 230. In addition, the external computing device 250 may predict the cardiopulmonary function index of the cardiopulmonary function measurement-target person 120 from the motion information, personal information and/or oxygen saturation information of the cardiopulmonary function measurement-target person 120. In addition, the predicted cardiopulmonary function index may be displayed on the display 252 of the external computing device 250 or may be transmitted to the gait measurement device 210 and/or the oxygen saturation measurement device 230.

In an embodiment, the external computing device 250 may be communicatively connected to the server 260 to transmit, to the server 260, the motion information, personal information and/or oxygen saturation information of the cardiopulmonary function measurement-target person 120. In addition, the external computing device 250 may obtain, from the server 260, the cardiopulmonary function index of the cardiopulmonary function measurement-target person 120 predicted by the server 260.

The display 252 may display the motion information, personal information, oxygen saturation information, and/or the predicted value of cardiopulmonary function index of the cardiopulmonary function measurement-target person 120.

In the memory 254, a program for controlling operation of the external computing device 250 may be stored. In the memory 254, according to motion information, personal information, and/or oxygen saturation information of the cardiopulmonary function measurement-target person 120 obtained from the gait measurement device 210 and/or the oxygen saturation measurement device 230, a program for determining the predicted value of the cardiopulmonary function index of the cardiopulmonary function measurement-target person 120 may be stored. In addition, in the memory 254, the motion information, personal information, oxygen saturation information, and/or the predicted value of the cardiopulmonary function index of the cardiopulmonary function measurement-target person 120 may be stored.

The processor 256 may perform instructions of the program for controlling operation of the external computing device 250. The processor 256 may perform instructions of the program of the predicted value of the cardiopulmonary function index of the cardiopulmonary function measurement-target person 120, according to the motion information, personal information, and/or oxygen saturation information of the cardiopulmonary function measurement-target person 120.

By the communication interface 258, the external computing device 250 may be communicatively connected to the gait measurement device 210, the oxygen saturation measurement device 230, and/or the server 260. Accordingly, the external computing device 250 may transmit setting information for measurement of motion information and/or oxygen saturation information to the gait measurement device 210 and/or the oxygen saturation measurement device 230 through the communication interface 258. In addition, the motion information and/or oxygen saturation information generated based on the setting information may be transmitted from the gait measurement device 210 and/or the oxygen saturation measurement device 230 to the external computing device 250 through the communication interface 258.

The server 260 may include a memory 262, a processor 264, and a communication interface 266. In some embodiments, the server 260 may additionally include components necessary for prediction of the cardiopulmonary function index.

In the memory 262, a program necessary to derive the server 260 may be stored. The memory 262 may store a cardiopulmonary function prediction database including statistical data for prediction and determination of the cardiopulmonary function index and at least one program for performing prediction and determination of the cardiopulmonary function index. In addition, the memory 262 may store the motion information, cardiopulmonary function index, personal information and oxygen saturation information of the cardiopulmonary function measurement-target person 120.

By the processor 264, an instruction of at least one program for performing prediction and determination of the cardiopulmonary function index stored in the memory 262 may be performed. By the processor 264, the cardiopulmonary function index of the cardiopulmonary function measurement-target person 120 may be determined from the motion information, personal information and/or oxygen saturation information of the cardiopulmonary function measurement-target person 120 received from the external computing device 250 through the communication interface 266. In addition, by the processor 264, the cardiopulmonary function index of the cardiopulmonary function measurement-target person 120 may be transmitted to the external computing device 250 through the communication interface 266.

The processor 264 may determine the cardiopulmonary function index of the cardiopulmonary function measurement-target person 120 from the motion information, personal information and/or oxygen saturation information of the cardiopulmonary function measurement-target person 120, based on the cardiopulmonary function prediction database stored in the memory 262. The processor 264 may update the cardiopulmonary function prediction database stored in the memory 262. Accordingly, the processor 264 may predict the cardiopulmonary function index of the cardiopulmonary function measurement-target person 120 based on the updated latest cardiopulmonary function prediction database.

The server 260 may include a communication interface 266 to receive and transmit data from and to the external computing device 250. The communication interface 266 may be implemented according to the communication standard such as mobile communication, Bluetooth, Wi-Fi, infrared data association (IrDA), WiMAX and the like.

The server 260 may receive, from the external computing device 250, the motion information, personal information and/or oxygen saturation information of the cardiopulmonary function measurement-target person 120 through the communication interface 266. In addition, the server 260 may transmit, to the external computing device 250, the cardiopulmonary function index of the cardiopulmonary function measurement-target person 120 and the medical information based on the cardiopulmonary function index through the communication interface 266. In addition, the server 260 may transmit a cardiopulmonary function prediction data set to the external computing device 250 through the communication interface 266.

The server 260 may include an additional processor for performing an instruction necessary for cardiopulmonary function prediction, in addition to the processor 264. In addition, the server 260 may perform an instruction for determination of the cardiopulmonary function index using the processor outside the server 260.

In an embodiment, the server 260 may be directly connected to the gait measurement device 210 and/or the oxygen saturation measurement device 230, without being connected to the external computing device 250. Accordingly, the server 260 may directly receive information necessary for prediction of the cardiopulmonary function index from the gait measurement device 210 and/or the oxygen saturation measurement device 230. In addition, the server 260 may directly transmit the predicted cardiopulmonary function index to the gait measurement device 210 and/or the oxygen saturation measurement device 230.

In an embodiment, the cardiopulmonary function index of the cardiopulmonary function measurement-target person 120 and information necessary for prediction of the cardiopulmonary function index may be stored only in the server 260 of the cardiopulmonary function measurement system 200, thereby preventing leakage of the personal information of the cardiopulmonary function measurement-target person 120.

In an embodiment, a person skilled in the art may easily design the cardiopulmonary function measurement system 200 to predict the cardiopulmonary function index of the cardiopulmonary function measurement-target person 120 in one or more of the gait measurement device 210, the oxygen saturation measurement device 230, the external computing device 250 and the server 260. Accordingly, although the gait measurement device 100 performs all functions necessary for prediction of the cardiopulmonary function index in FIG. 1, in the cardiopulmonary function measurement system 200 of FIG. 2, the functions of the gait measurement device 100 may be distributed to the gait measurement device 210, the oxygen saturation measurement device 230, the external computing device 250 and the server 260.

In addition, a person skilled in the art may easily design the cardiopulmonary function measurement system 200 to transmit information necessary for prediction of the cardiopulmonary function index of the cardiopulmonary function measurement-target person 120 between the gait measurement device 210, the oxygen saturation measurement device 230, the external computing device 250, and the server 260. In addition, the cardiopulmonary function measurement system 200 may include an additional device for obtaining information necessary for prediction of the cardiopulmonary function index.

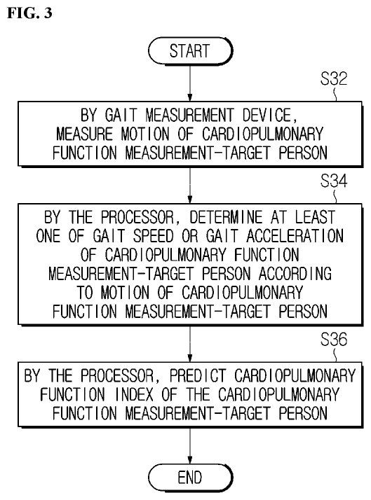

FIG. 3 shows an embodiment of a cardiopulmonary function index prediction method performed by the gait measurement device 100.

In step S32, by a motion sensor, motion of a cardiopulmonary function measurement-target person is measured.

In an embodiment, the motion sensor may be implemented by a 2D LIDAR, and two-dimensional motion of the cardiopulmonary function measurement-target person may be measured using the 2D LIDAR.

In an embodiment, an effective measurement range of the motion sensor may be set. In addition, motion of the cardiopulmonary function measurement-target person within the effective measurement range may be measured.

In an embodiment, one of a gait speed measurement mode in which the gait speed of the cardiopulmonary function measurement-target person is measured and a gait analysis mode in which motion information according to gait analysis of the cardiopulmonary function measurement-target person is additionally obtained in addition to the gait speed may be selected. In addition, according to the selected measurement mode, the motion information of the cardiopulmonary function measurement-target person is generated.

In step S34, by the processor, the gait speed of the cardiopulmonary function measurement-target person is determined according to motion of the cardiopulmonary function measurement-target person. In addition, according to the gait speed of the cardiopulmonary function measurement-target person, the gait acceleration of the cardiopulmonary function measurement-target person may be determined.

In an embodiment, based on two-dimensional motion of the cardiopulmonary function measurement-target person, at least one of a vertical component or horizontal component of the gait speed or a vertical component or horizontal component of the gait acceleration may be measured.

In an embodiment, a time-based graph of at least one of the gait speed or gait acceleration of the cardiopulmonary function measurement-target person may be generated.

In step S36, by the processor, based on the gait speed of the cardiopulmonary function measurement-target person, the cardiopulmonary function index of the cardiopulmonary function measurement-target person is predicted. In addition, the cardiopulmonary function index may be predicted by further considering the gait acceleration of the cardiopulmonary function measurement-target person.

In an embodiment, by the processor, a gait speed parameter indicating the gait speed may be determined. Similarly, a gait acceleration parameter indicating the gait acceleration may be determined. The gait speed parameter represents the gait speed of a specific section, and the gait acceleration parameter represents the gait acceleration of a specific section.

In addition, by the processor, the predicted value of the cardiopulmonary function index may be determined according to the gait speed parameter and/or the gait acceleration parameter. According to a gait speed-cardiopulmonary function correlation function, the predicted value of the cardiopulmonary function index may be determined based on the gait speed parameter. In addition, according to the correlation function of the gait acceleration-cardiopulmonary function, the predicted value of the cardiopulmonary function index may be determined based on the gait acceleration parameter.

In an embodiment, by the processor, according to at least one of the vertical component or horizontal component of the gait speed or the vertical component or horizontal component of the gait acceleration, the predicted value of the cardiopulmonary function index may be determined.

In an embodiment, by further considering the personal information of the cardiopulmonary function measurement-target person in addition to the motion information such as the gait speed, the cardiopulmonary function index of the cardiopulmonary function measurement-target person may be predicted.

In an embodiment, according to a gait pattern shown in the time-based graph of the gait speed and/or the time-based graph of the gait acceleration, the cardiopulmonary function index of the cardiopulmonary function measurement-target person may be predicted.

In an embodiment, by an oxygen saturation measurement device, the oxygen saturation of the cardiopulmonary function measurement-target person may be measured. In addition, by further considering the oxygen saturation, the cardiopulmonary function index of the cardiopulmonary function measurement-target person may be predicted.

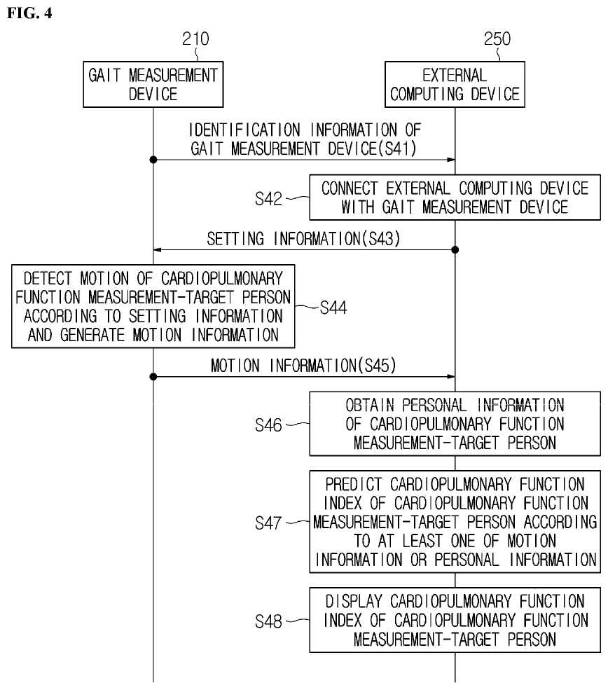

FIG. 4 shows an embodiment of a cardiopulmonary function index prediction method according to a cardiopulmonary function measurement system including the gait measurement device 210 and the external computing device 250.

In step S41, identification information of the gait measurement device 210 is transmitted from the gait measurement device 210 to the external computing device 250.

In step S42, whether the identification information of the gait measurement device 210 is valid is determined by the external computing device 250. When the identification information of the gait measurement device 210 is valid, the external computing device 250 and the gait measurement device 210 are communicatively connected.

In step S43, setting information related to motion detection of the cardiopulmonary function measurement-target person 120 is transmitted from the external computing device 250 to the gait measurement device 210.

In step S44, the gait measurement device 210 detects motion of the cardiopulmonary function measurement-target person 120 according to the transmitted setting information. The setting information may include a measurement direction, a measurement range and an effective measurement range. In addition, the gait measurement device 210 generates motion information including at least one of a position, a gait speed or gait acceleration.

In step S45, the motion information of the cardiopulmonary function measurement-target person 120 is transmitted from the gait measurement device 210 to the external computing device 250.

In step S46, the personal information of the cardiopulmonary function measurement-target person 120 is input to the external computing device 250.

In step S47, by the external computing device 250, the cardiopulmonary function index of the cardiopulmonary function measurement-target person 120 is calculated from the motion information and personal information of the cardiopulmonary function measurement-target person 120.

In step S48, the cardiopulmonary function index of the cardiopulmonary function measurement-target person 120 is displayed by the display 252 of the external computing device 250.

FIG. 5 shows an embodiment of a cardiopulmonary function index prediction method according to the cardiopulmonary function measurement system including the gait measurement device 210, the external computing device 250 and the server 260.

In step S51, the identification information of the gait measurement device 210 is transmitted from the gait measurement device 210 to the external computing device 250.

In step S52, whether the identification information of the gait measurement device 210 is valid is determined by the external computing device 250. When the identification information of the gait measurement device 210 is valid, the external computing device 250 and the gait measurement device 210 are communicatively connected.

In step S53, setting information related to motion detection of the cardiopulmonary function measurement-target person 120 is transmitted from the external computing device 250 to the gait measurement device 210.

In step S54, the gait measurement device 210 detects motion of the cardiopulmonary function measurement-target person 120 according to the transmitted setting information. In addition, the gait measurement device 210 generates motion information including a position, a gait speed or a gait acceleration.

In step S55, the motion information of the cardiopulmonary function measurement-target person 120 is transmitted from the gait measurement device 210 to the external computing device 250.

In step S56, the personal information of the cardiopulmonary function measurement-target person 120 is input to the external computing device 250.

In step S57, the motion information and personal information of the cardiopulmonary function measurement-target person 120 are transmitted from the external computing device 250 to the server 260. Additionally, other information necessary for calculation of the cardiopulmonary function index may be transmitted from the external computing device 250 to the server 260.

In step S58, by the server 260, the cardiopulmonary function index of the cardiopulmonary function measurement-target person 120 is calculated from the motion information and personal information of the cardiopulmonary function measurement-target person 120.

In S59, the cardiopulmonary function index of the cardiopulmonary function measurement-target person 120 is transmitted from the server 260 to the external computing device 250.

In step S60, the cardiopulmonary function index of the cardiopulmonary function measurement-target person 120 is displayed by the display 252 of the external computing device 250.

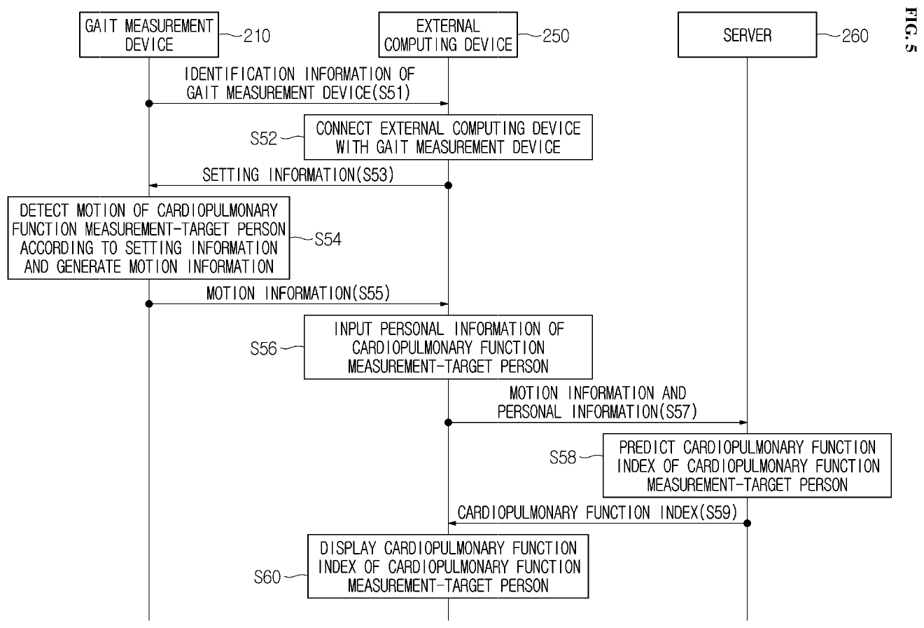

FIG. 6 shows an embodiment of a cardiopulmonary function index prediction method according to the cardiopulmonary function measurement system including the gait measurement device 210, the oxygen saturation measurement device 230, the external computing device 250 and the server 260.

In step S61, identification information of the gait measurement device 210 and the oxygen saturation measurement device 230 is transmitted from the gait measurement device 210 and the oxygen saturation measurement device 230 to the external computing device 250.

In step S62, whether the identification information of the gait measurement device 210 and the oxygen saturation measurement device 230 is valid is determined by the external computing device 250. When the identification information of the gait measurement device 210 and the oxygen saturation measurement device 230 is valid, the external computing device 250, the gait measurement device 210 and the oxygen saturation measurement device 230 are communicatively connected.

In step S63, setting information related to motion detection and oxygen saturation measurement of the cardiopulmonary function measurement-target person 120 is transmitted from the external computing device 250 to the gait measurement device 210. In addition, setting information related to measurement of the oxygen saturation of the cardiopulmonary function measurement-target person 120 is transmitted from the external computing device 250 to the oxygen saturation measurement device 230.

In step S64, the gait measurement device 210 detects motion of the cardiopulmonary function measurement-target person 120 according to the transmitted setting information. In addition, the gait measurement device 210 generates motion information including at least one of a position, a gait speed or a gait acceleration.

In step S65, motion information of the cardiopulmonary function measurement-target person 120 is transmitted from the gait measurement device 210 to the external computing device 250.

In step S66, the oxygen saturation measurement device 230 measures the oxygen saturation of the cardiopulmonary function measurement-target person 120 according to the transmitted setting information. In addition, the oxygen saturation measurement device 230 generates oxygen saturation information related to oxygen saturation.

In step S67, the oxygen saturation information of the cardiopulmonary function measurement-target person 120 is transmitted from the oxygen saturation measurement device 230 to the external computing device 250.

In step S68, the personal information of the cardiopulmonary function measurement-target person 120 is input to the external computing device 250.

In step S69, the motion information, oxygen saturation information and personal information of the cardiopulmonary function measurement-target person 120 are transmitted from the external computing device 250 to the server 260. Additionally, other information necessary for calculation of the cardiopulmonary function index may be transmitted from the external computing device 250 to the server 260.

In step S70, by the server 260, the cardiopulmonary function index of the cardiopulmonary function measurement-target person 120 is calculated from the motion information, oxygen saturation information and personal information of the cardiopulmonary function measurement-target person 120.

In step S71, the cardiopulmonary function index of the cardiopulmonary function measurement-target person 120 is transmitted from the server 260 to the external computing device 250.

In step S72, the cardiopulmonary function index of the cardiopulmonary function measurement-target person 120 is displayed by the display 252 of the external computing device 250.

The embodiments of FIGS. 3 to 6 are only examples and the cardiopulmonary function index prediction method according to the present disclosure is not limited to the embodiments of FIGS. 3 to 6. Those skilled in the art of the cardiopulmonary disease may easily change the embodiments of FIGS. 3 to 6 with reference to the function and operation performed by the gait measurement device 100 and the cardiopulmonary function index prediction system 200 described in FIGS. 1 and 2.

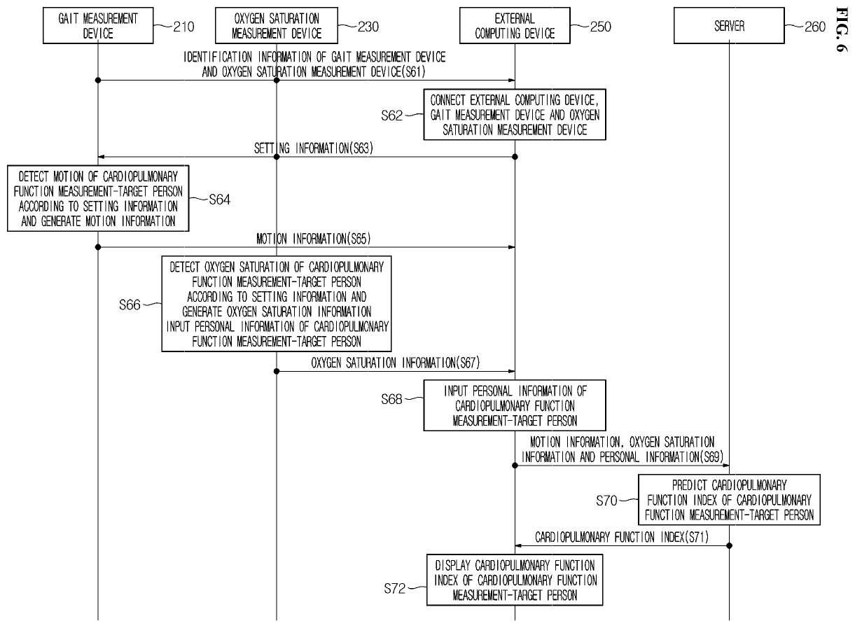

FIG. 7 shows an embodiment of a setting item 710 of a gait speed measurement item 700 shown on the displays 102 and 252, in a program performed in the gait measurement device 100 or the external computing device 250.

The setting item 710 includes a measurement range setting item 720 and a measurement method setting item 730.

In the measurement range setting item 720, setting values related to the measurement range of the motion sensors 106 and 216 are displayed. Specifically, the measurement range setting item 720 include motion sensor information 721, measurement direction information 722, distance offset information 723, and effective measurement range length information 724 and effective measurement range width information 725 respectively indicating the length and width of the effective measurement range.

The motion sensor information 721 indicates the type of the motion sensors 106 and 216. In addition, according to the type of the motion sensors 106 and 216, the measurement range of the motion sensors 106 and 216 is determined. The measurement range indicates a maximum range in which motion of the object of the motion sensors 106 and 216 may be detected.

An effective measurement range in which motion of the cardiopulmonary function measurement-target person 120 is recorded is determined within the measurement range. When all motions of the measurement range is recorded, noise may occur in motion information. Therefore, by setting the effective measurement range and allowing the cardiopulmonary function measurement-target person 120 to walk within the effective measurement range, motion information of the cardiopulmonary function measurement-target person 120 may be efficiently generated.

The effective measurement range may be determined according to measurement direction information 722, distance offset information 723, effective measurement range length information 724 and effective measurement range width information 725. Although the effective measurement range is set in a rectangular shape in the present disclosure, it may be set in the other shapes according to an embodiment. The measurement direction information 722, the distance offset information 723, the effective measurement range length information 724 and the effective measurement range width information 725 may be modified by user input.

The measurement direction information 722 indicates the measurement direction of the motion sensors 106 and 216. In addition, the distance offset information 723 indicates a minimum vertical distance of the motion sensors 106 and 216 and the effective measurement range. In addition, the effective measurement range length information 724 and the effective measurement range width information 725 indicate the length and width of the effective measurement range, respectively. The effective measurement range according to the measurement direction information 722, the distance offset information 723, the effective measurement range length information 724 and the effective measurement range width information 725 is confirmed in a data confirmation window 750.

The measurement method setting item 730 indicates a method of determining motion information from motion of the cardiopulmonary function measurement-target person 120. Specifically, the measurement method setting item 730 may include a measurement mode item 731, a gait speed measurement method item 732, and a measurement start option item 740.

A travel distance calculation method item 731 represents a method of calculating the travel distance of the cardiopulmonary function measurement-target person 120. In the present disclosure, in the measurement mode item 731, one of a gait speed measurement mode in which the gait speed of the cardiopulmonary function measurement-target person 120 is measured and a gait analysis mode in which motion information according to gait analysis of the cardiopulmonary function measurement-target person 120 is additionally obtained in addition to the gait speed may be selected. According to the gait analysis mode of the cardiopulmonary function measurement-target person 120, motion information such as step length, step width, stride length, number of steps, gait distance, gait speed, gait acceleration, etc. is calculated. According to the gait speed measurement mode, only the gait speed of the cardiopulmonary function measurement-target person 120 is measured.

The gait speed measurement method item 732 represents the gait pattern of the cardiopulmonary function measurement-target person 120 for gait speed measurement. In the present disclosure, in the gait speed measurement method item 732, one of a one-way measurement method item 733 and a round-trip measurement method 736 may be selected.

According to the one-way measurement method, a time required for the cardiopulmonary function measurement-target person 120 to travel by a predetermined measurement distance in one direction is measured. In addition, the gait speed of the cardiopulmonary function measurement-target person 120 is measured from a gait speed measurement section in which a change in gait speed is small. Therefore, a section in which the magnitude of the gait acceleration is greater than a predetermined acceleration threshold is excluded from the gait speed measurement section. The predetermined measurement distance and the predetermined acceleration threshold may be determined according to a measurement distance item 734 and an acceleration threshold item 735. In addition, the measurement distance item 734 and the acceleration threshold item 735 may be determined according to user input.

The round-trip measurement method 736 includes a travel distance-based measurement method item 737 and a time-based measurement method item 738. According to the travel distance-based measurement method, the gait speed is measured based on a time required for the cardiopulmonary function measurement-target person 120 to complete walking of a predetermined round-trip distance. According to the time-based measurement method, the gait speed is measured based on a round-trip travel distance of the cardiopulmonary function measurement-target person 120 for a predetermined measurement time. The travel distance-based measurement method item 737 represents a predetermined round-trip travel distance, and the time-based measurement method item 738 represents a predetermined measurement time. In addition, the predetermined round-trip travel distance and the predetermined measurement time may be determined according to user input.

The measurement start option item 740 indicates a measurement start condition. In the present disclosure, in the measurement start option item 740, one of an immediate start option, a motion detection start option and a specific distance start option may be selected. According to the immediate start option, when a user activates a start 770, motion measurement of the cardiopulmonary function measurement-target person 120 starts. According to the motion detection start option, when motion of the cardiopulmonary function measurement-target person 120 is detected, motion measurement of the cardiopulmonary function measurement-target person 120 starts. According to the specific distance start option, when detecting the cardiopulmonary function measurement-target person 120 is located at a specific distance, motion measurement of the cardiopulmonary function measurement-target person 120 starts.

In an embodiment, the setting item 710 may include a motion information display 750. On the a motion information display 750, motion of the cardiopulmonary function measurement-target person 120 is displayed.

In an embodiment, the setting item 710 may further display a disconnect 750 and a start 770. When the start tracking 770 is activated by a user, motion of the cardiopulmonary function measurement-target person 120 is recorded in the gait measurement device 100 or the external computing device 250. By the disconnect 750 is activated by a user, connection between the external computing device 250 and the gait measurement device 210 is finished.

FIG. 8 shows an embodiment of a gait speed measurement item 810 of the gait speed measurement item 700 shown in the displays 102 and 252, in a program performed in the gait measurement device 100 or the external computing device 250.

The gait speed measurement item 810 may include a gait distance-time graph 820, a personal information item 830 and a measurement result item 840.

The gait distance-time graph 820 indicates the gait distance of the cardiopulmonary function measurement-target person 120 over time. According to the gait distance-time graph 820, the gait pattern of the cardiopulmonary function measurement-target person 120 is visually provided.

The personal information item 830 indicates the personal information of the cardiopulmonary function measurement-target person 120. The personal information of the cardiopulmonary function measurement-target person 120 may be determined by user input.

The measurement result item 840 indicates motion information according to the gait test of the cardiopulmonary function measurement-target person 120. The measurement result item 840 may indicate a total gait distance, a total time and a gait speed. In addition, a measurement result required for cardiopulmonary function prediction may be additionally displayed on the measurement result item 840.

An analysis result item 850 indicates an analysis result of the motion information of the cardiopulmonary function measurement-target person 120. The analysis result item 850 may indicate an average step length, an average step width, an average stride length, average number of steps, a gait speed, etc. In addition, an analysis result required for cardiopulmonary function prediction may be additionally displayed in the analysis result item 850.

Although the measurement result item 840 and the analysis result item 850 are divisionally shown in FIG. 8, only one item obtained by combining the measurement result item 840 and the analysis result item 850 may be displayed in the gait speed measurement item 810.

In an embodiment, the gait speed measurement item 810 may include a motion information display 860. On the motion information display 860, a motion result of the cardiopulmonary function measurement-target person 120 is displayed. In addition, the gait speed measurement item 810 may include a gait analysis display 860. On the gait analysis display 860, the gait of the cardiopulmonary function measurement-target person 120 is displayed.

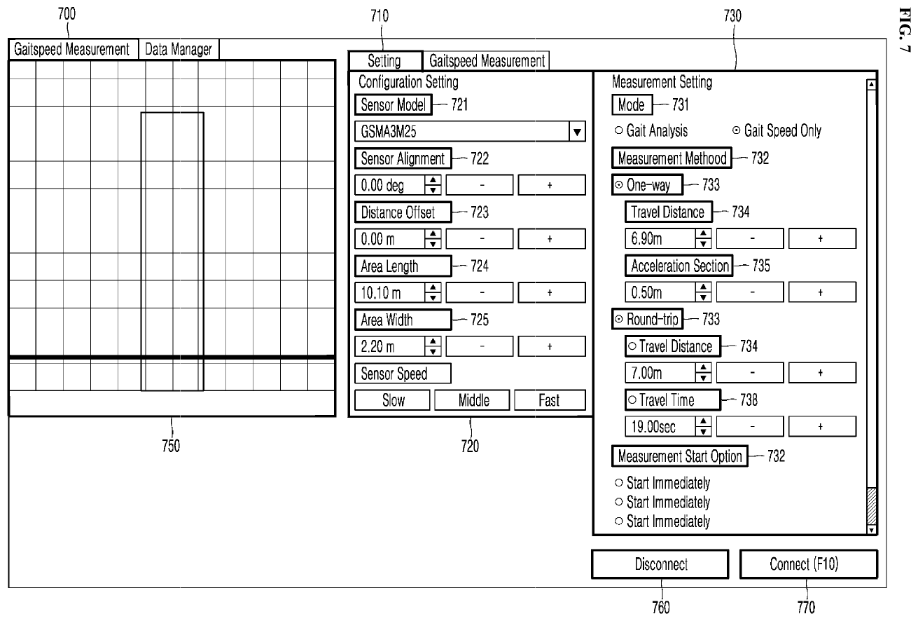

FIG. 9 shows an embodiment of a data management item 900 shown on the displays 102 and 252, in a program performed by the gait measurement device 100 or the external computing device 250.

The data management item 900 may include a data field 910, a personal information item 920, a measurement result item 930, an analysis result item 940, a gait distance-time graph 950 and a walking path image 960.

In the data field 910, data sets representing the gait test result of the cardiopulmonary function measurement-target person 120 are aligned. The data sets of the data field 910 include the name, gait test identification number, gait speed, total gait distance, gait time and measurement time of the cardiopulmonary function measurement-target person 120.

By user input, a data set of the data field 910 may be selected. In an embodiment, a personal information item 920, a measurement result item 930, a gait distance-time graph 940 and a walking path image 950 indicating data on the selected data set is displayed on the right side of the data field 910.

The personal information item 920 indicates the personal information of the cardiopulmonary function measurement-target person 120. The personal information of the cardiopulmonary function measurement-target person 120 may be modified by user input.

The measurement result item 930 indicates motion information of the gait test of the cardiopulmonary function measurement-target person 120. The measurement result item 840 may indicate a total gait distance, a total time, a gait speed, a gait speed calculation method, an acceleration threshold and a measurement time, etc. In addition, a measurement result required for cardiopulmonary function prediction may be additionally displayed in the measurement result item 930.

The analysis result item 940 indicates an analysis result of motion information according to the gait test of the cardiopulmonary function measurement-target person 120. The analysis result item 940 may include a measurement mode, a step length, a step width, a stride length, the number of steps, etc. In addition, an analysis result required for cardiopulmonary function prediction may be additionally displayed in the analysis result item 930.

Although the measurement result item 930 and the analysis result item 940 are divisionally shown in FIG. 9, according to another embodiment, only one item obtained by combining the measurement result item 930 and the analysis result item 940 may be displayed in the data management item 900.

The gait distance-time graph 950 indicates the gait distance of the cardiopulmonary function measurement-target person 120 over time, similarly to the gait distance-time graph 820 of FIG. 8. According to the gait distance-time graph 950, the gait pattern of the cardiopulmonary function measurement-target person 120 is visually provided.

The walking path image 960 visually represents the walking path of the cardiopulmonary function measurement-target person 120.

An embodiment of the program described in FIGS. 7 to 9 is only an example, and the program in which the cardiopulmonary function prediction method of the present disclosure is implemented is not limited to the embodiments of FIGS. 7 to 9. In addition, a person skilled in the art may easily change the configuration of the program of FIGS. 7 to 9, based on the cardiopulmonary function prediction method and device of FIGS. 1 to 6.

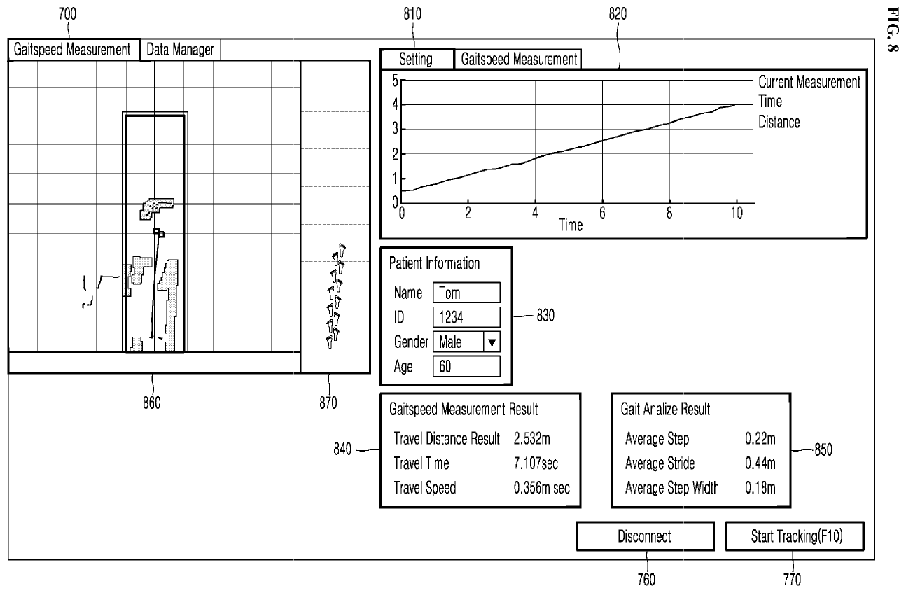

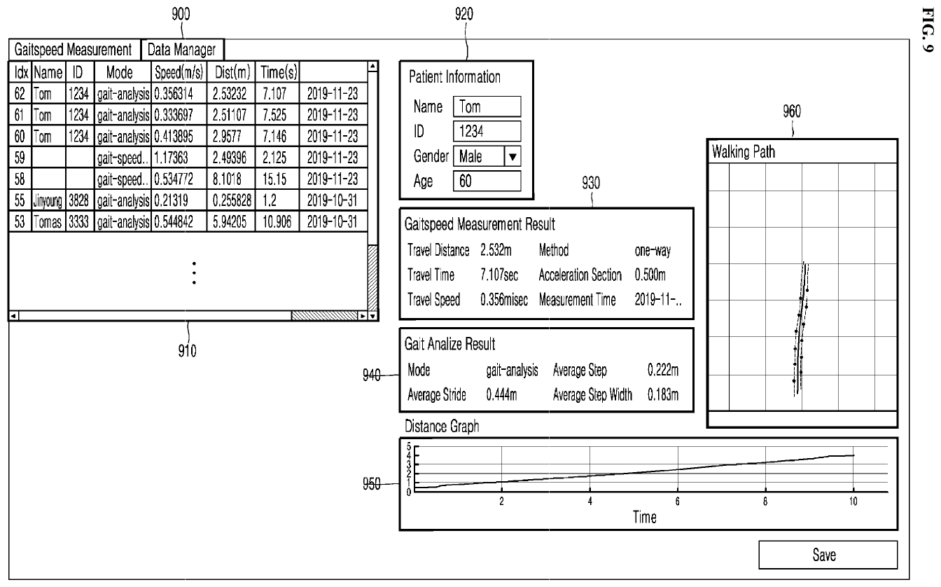

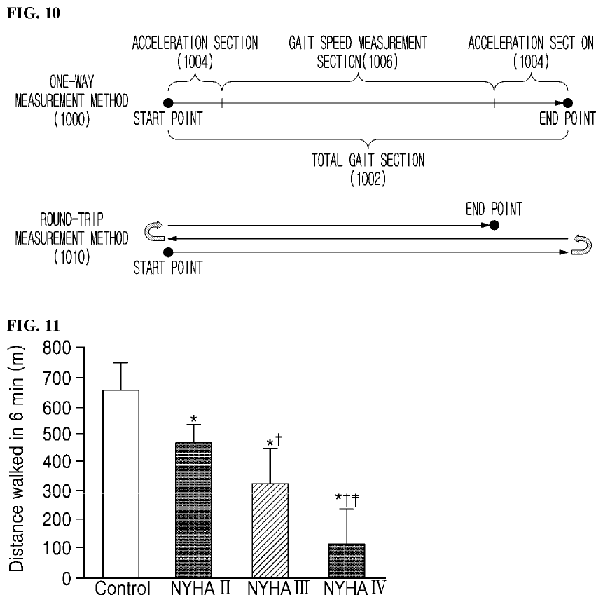

FIG. 10 shows a method of measuring a gait speed according to an embodiment.

According to the one-way measurement method 1100, the cardiopulmonary function measurement-target person 120 walks in a total walking section 1002. In addition, the gait speed of the cardiopulmonary function measurement-target person 120 is measured from a gait speed measurement section 1006 in which a change in gait speed is small. Therefore, an acceleration section 1004 in which the magnitude of the gait acceleration is less than a predetermined acceleration threshold is not included in the gait speed measurement section 1006. Accordingly, the gait speed may be determined according to the gait time and gait distance of the gait speed measurement section 1006.

According to the round-trip measurement method 1010, the cardiopulmonary function measurement-target person 120 goes and returns in a specific section. In the round-trip measurement method 1010, the gait speed may be calculated based on a travel distance or a gait time.

According to the travel distance-based measurement method, the cardiopulmonary function measurement-target person 120 walks in a round-trip section having a predetermined distance. In addition, a time when the walking of the round-trip section having the predetermined distance is completed, the gait speed of the cardiopulmonary function measurement-target person 120 is calculated. According to the time-based measurement method, the cardiopulmonary function measurement-target person 120 walks in the round-trip section for a predetermined time. In addition, according to the length of the round-trip section in which the person has walked for the predetermined time, the gait speed of the cardiopulmonary function measurement-target person 120 is calculated.

FIG. 11 shows a graph showing a gait distance of each group according to each New York Heart Association (NYHA) functional classification according to a 6-minute gait test. The NYHA functional classification is a method of classifying the severity of heart failure. According to the NYHA functional classification, patients may be classified into four groups (NYHA Class I, NYHA Class II, NYHA Class III, NYHA Class VI) according to the severity of the heart failure. NYHA Class I includes patients without the symptoms of heart failure, and the more severe the symptom of heart failure, the higher the class.

According to the graph of FIG. 11, patients classified as a control group, that is, NYHA Class I, walk by 600 meters to 700 meters for 6 minutes. In addition, patients classified as NYHA Class II walk by 400 meters to 500 meters for 6 minutes. In addition, patients classified as NYHA Class III walk by 300 meters for 6 minutes. In addition, patients classified as NYHA Class VI with the most severe heart failure walk by about 100 meters for 6 minutes.

Accordingly, it can be seen that the heart failure and the gait speed are closely related. Therefore, the severity of heart failure may be predicted according to the gait speed.

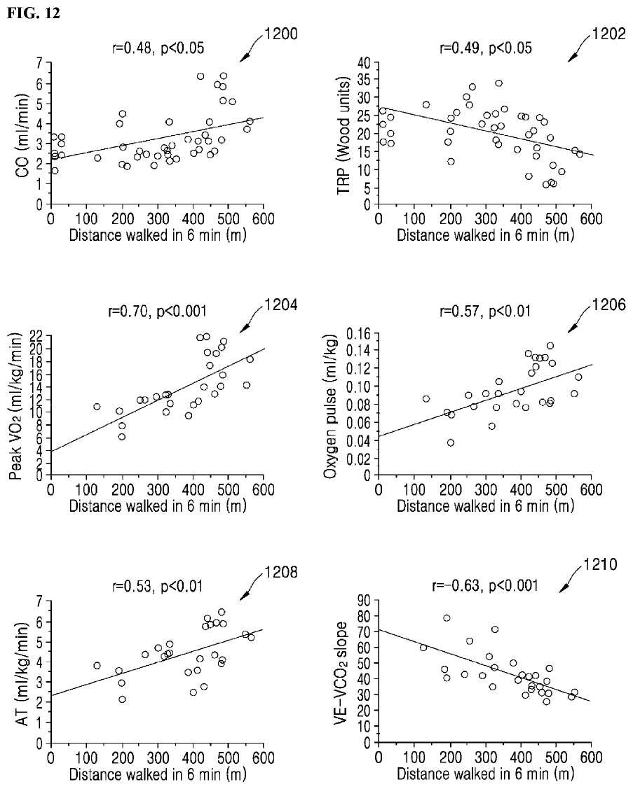

FIG. 12 shows graphs illustrating correlation between a gait distance according to a 6-minute gait test and a cardiac output (CO), TPR (Total Pulmonary Resistance), Peak VO2 (peak exercise oxygen consumption), AT (anaerobic threshold), Oxygen Pulse and VE-VCO2 slope (regression slope relating minute ventilation to carbon dioxide output).

A first graph 1200 shows a relationship between the gait distance according to the 6-minute gait test and the CO (Cardiac Output). The CO means refers to the amount of blood output from the heart for 1 minute. According to the first graph 1200, it can be seen that the CO statistically increases as the gait distance increases. Therefore, it can be predicted that the larger the gait speed, the larger the CO.

A second graph 1202 shows a relationship between the gait distance according to the 6-minute gait test and the TPR (Total Pulmonary Resistance). The TPR means refers to a required pressure difference between an oral cavity and a pleural surface of a lung for causing air flow in the oral cavity. According to the second graph 1202, it can be seen that the TPR statistically decreases as the gait distance increases. Therefore, it can be predicted that the larger the gait speed, the smaller the TPR.

A third graph 1204 shows a relationship between the gait distance according to the 6-minute gait test and the Peak VO2 (peak exercise oxygen consumption). The Peak VO2 refers to the peak amount of oxygen which can be consumed by the human body during exercise. According to the third graph 1204, it can be seen that the Peak VO2 statistically increases as the gait distance increases. Therefore, it can be predicted that the larger the gait speed, the larger the Peak VO2.

A fourth graph 1206 shows a relationship between the gait distance according to the 6-minute gait test and the oxygen pulse. The oxygen pulse refers to a value obtained by dividing the amount of oxygen absorbed by the lung for 1 minute by the number of pulses for 1 minutes. According to the third graph 1206, it can be seen that the oxygen pulse statistically increases as the gait distance increases. Therefore, it can be predicted that the larger the gait speed, the larger the oxygen pulse.

A fifth graph 1208 shows a relationship between the gait distance according to the 6-minute test and the AT (anaerobic threshold). The AT refers to oxygen consumption of an exercise intensity at which anaerobic metabolism begins. According to the fifth graph 1208, it can be seen that the AT statistically increases as the gait distance increases. Therefore, it can be predicted that the larger the gait speed, the larger the AT.

A sixth graph 1210 shows a relationship between the gait distance according to the 6-minute gait test and the VE-VCO2 slope (regression slope relating minute ventilation to carbon dioxide output). The VE-VCO2 slope refers to the magnitude of the ratio of the amount of carbon dioxide output from the body to the amount of air circulated for 1 minute. According to the sixth graph 1210, it can be seen that the VE-VCO2 slope statistically decreases as the gait distance increases. Therefore, it can be predicted that the larger the gait speed, the smaller the VE-VCO2 slope.

Cardiopulmonary function indices introduced in FIGS. 11 and 12 are important in diagnosis of cardiopulmonary diseases. Accordingly, according to the relationship between the cardiopulmonary function indices and the gait speed, the cardiopulmonary function indices may be predicted from the gait speed. Although not introduced in FIGS. 11 and 12, other cardiopulmonary function indices having a statistical relationship with the gait speed may also be predicted from the gait speed.

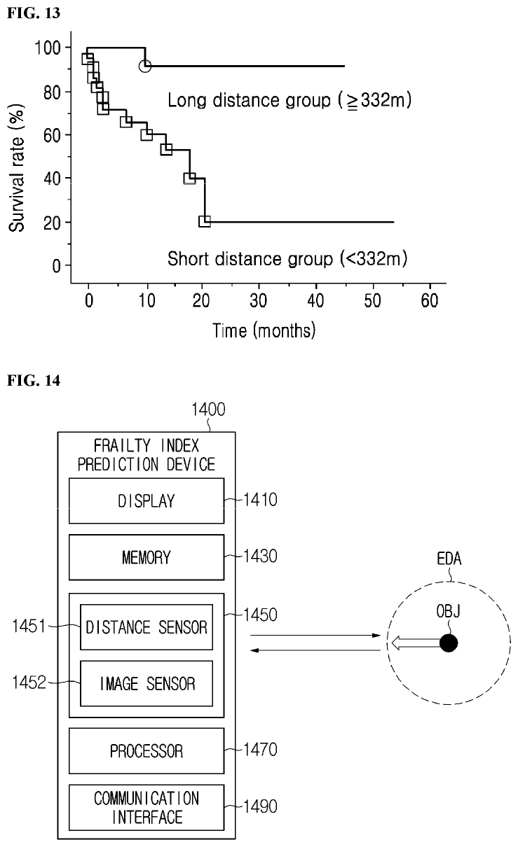

FIG. 13 shows a graph of a Kaplan-Meier survival curve of two groups according to a 6-minute gait test. The Kaplan-Meier survival curve represents a survival rate of an experimental group over time.

In FIG. 13, according to the 6-minutes gait test, PPH (primary pulmonary hypertension) patients are classified into an upper gait distance group and a lower gait distance group. The upper gait distance group has a survival rate of greater than 90% during 50 months. However, the lower gait distance group has the survival rate rapidly decreasing over time. In particular, the survival rate of the lower gait distance group rapidly decreases to 20% after 20 months. Accordingly, by measuring the gait speed of the PPH patient, the survival rate of the patient is significantly predicted.

Like the relationship between the survival rate according to the PPH and the gait speed shown in FIG. 13, a relationship between a survival rate according to another cardiopulmonary disease and the gait speed may be derived. Accordingly, the survival rate of patients with other cardiopulmonary diseases may be predicted according to the gait speed.

Hereinafter, a frailty index prediction device and method according to embodiments of the present disclosure will be described in detail with reference to the accompanying drawings.

FIG. 14 is a view illustrating a frailty index prediction device according to an embodiment of the present disclosure.

Referring to FIG. 14, the frailty index prediction device 1400 may include a display 1410, a memory 1430, a sensor 1450, a processor 1470 and a communication interface 1490. In some embodiments, the frailty index prediction device 1400 may further include an additional component necessary to evaluate physical performance of a subject OBJ and to predict a frailty index.

The display 1410 may perform an output interface function for displaying motion information, physical performance evaluation result, frailty index prediction result, etc. of the subject OBJ. Here, the motion information may include body information regarding the pose or shape of the subject OBJ. In an example, the motion information may be estimated by receiving distance information and image information of the subject OBJ obtained through the sensor 1450 as input and executing an Artificial Neural Network (ANN). The physical performance evaluation result may include at least one physical performance parameter extracted from the motion information of the subject OBJ. For example, a physical performance evaluation result may include at least one of a gait speed, balance time, sit-to-stand time or TUG (timed-up-and-go) time of the subject OBJ. A frailty index prediction result may include at least one of a frailty index, a physiological age, sarcopenia or a fall-risk predicted from the physical performance parameter of the subject OBJ.

In an example, the display 1410 may be implemented as a touchscreen panel to perform an input interface function. For example, a user may input personal information such as age, gender and presence/absence of a disease of the subject OBJ through the display 1410. In addition, the user may input device setting information for physical performance evaluation and frailty index prediction through the display 1410.

The memory 1430 may store various types of data necessary for operation of the frailty index prediction device 1400. For example, the memory 1430 may store an operation program for driving the frailty index prediction device 1400. In addition, the memory 1430 may store sensing data indicating the body information of the subject OBJ and data regarding the physical performance evaluation result and the frailty index prediction result obtained based on the body information of the subject OBJ. In an example, the memory 1430 may include at least one of a non-volatile memory such as a NAND flash memory or a volatile memory such as a DRAM.

The sensor 1450 may obtain distance information and image information of the subject OBJ. In an example, a predetermined effective detection area EDA may be set for the sensor 1450. In this case, the sensor 1450 may obtain the distance information and image information of the subject OBJ within the EDA. The EDA may be adjusted by the user within a predetermined range determined according to the specification of the sensor 1450.

The sensor 1450 may include a distance sensor 1452 and an image sensor 1452.

The distance sensor 1452 outputs inspection waves of various waveforms, such as ultrasonic waves, infrared rays, lasers, etc. and detects the inspection waves reflected by the subject OBJ, thereby obtaining distance information regarding a distance and direction from an observation point to the subject OBJ. In an example, the distance sensor 1451 may include at least one of a ToF (Time of Flight) sensor, an ultrasonic sensor, a structured light sensor, an infrared sensor or a LiDAR sensor.

The image sensor 1452 may obtain image information of the subject OBJ, by detecting a light signal output from a light source and reflected by the subject OBJ. In this specification, the image sensor 1452 may be referred to as a color camera. In an example, the image sensor 1452 may include a light source, a pixel array and a light detection circuit. The light source may include a plurality of light emitting elements (for example, VCSEL (Vertical Cavity Surface Emitting Laser), LED, etc.) for outputting a light signal of a specific band wavelength. The pixel array includes a plurality of pixels, and each pixel may include a plurality of photo sensing elements (e.g., a photodiode, etc.) and a color filter (e.g., RGB filter).

In an example, the sensor 1450 may have a single camera structure including a single distance sensor and a single image sensor. In addition, since the distance sensor 1452 and the image sensor 1452 may be integrally implemented as one module, a separate test space for sensor installation does not need to be secured and a compact form factor may be achieved.

The processor 1470 may control overall operation of the frailty index prediction device 1400. In addition, the processor 1470 may evaluate the physical performance of the subject OBJ based on the body information of the subject OBJ obtained by the sensor 1450 and predict a frailty index. For example, the processor 1470 may obtain motion information such as pose or shape of the subject OBJ, based on the distance information and image information of the subject OBJ obtained by the sensor 1450. The processor 1470 may extract the physical performance parameter of the subject OBJ from the obtained motion information and predict the frailty index of the subject OBJ.

Meanwhile, the distance information obtained by the distance sensor 1451 may have an insufficient amount of data to extract the motion information of the subject OBJ. Accordingly, in an embodiment, the processor 1470 may up-sample the distance information of the subject OBJ, by fusing the distance information obtained by the distance sensor 1451 and the image information obtained by the image sensor 1452 using an artificial neural network (ANN). Here, the ANN may include a DNN (Deep Neural Network), a CNN (Convolutional Neural Network), a RNN (Recurrent Neural Network), or a GAN (Generative Adversarial Network), and may be implemented inside the processor 1470 or may be implemented as a separate processor outside the processor 1470.

The communication interface 1490 may perform a communication function for transmitting and receiving data to and from an external device. For example, the distance information and image information of the subject OBJ may be transmitted and received to and from the external device through the communication interface 1490. In addition, the physical performance parameter extraction result or frailty index prediction result of the subject OBJ, obtained based on the distance information and the image information, may be transmitted to and from the external device through the communication interface 1490. In addition, user information and various types of device setting values of the frailty index prediction device 1400 may be transmitted from and to the external device through the communication interface 1490.

As described above, the frailty index prediction device 1400 may extract the physical performance parameter of the subject OBJ from the distance information and image information of the subject OBJ obtained by the sensor 1450, and predict the frailty index based on the extracted physical performance parameter. In an example, the sensor 1450 may have a single camera structure including a single distance sensor 1451 and a single image sensor 1452. In addition, in an example, the processor 1470 may up-sample the distance information of the subject OBJ obtained by the distance sensor 1451 using the image information obtained by the image sensor 1452.

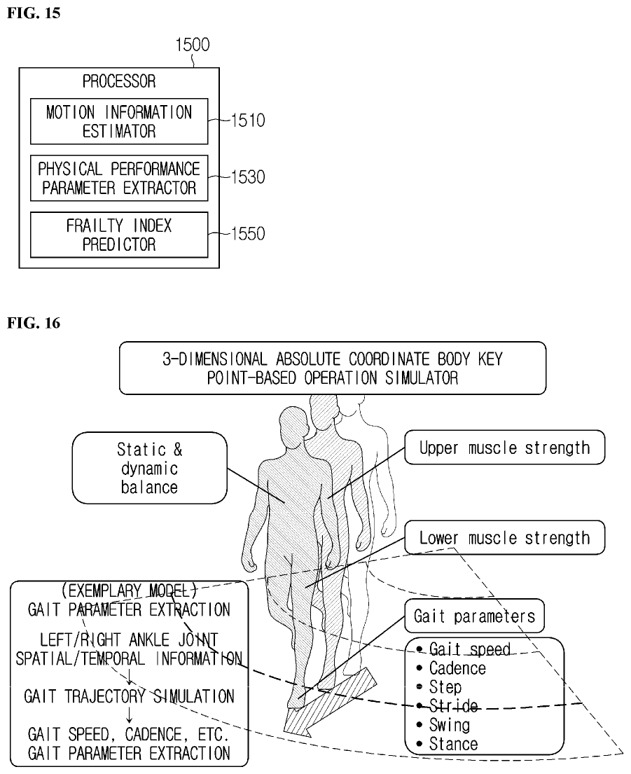

FIG. 15 is a view illustrating a configuration of a processor in a frailty index prediction device according to an embodiment of the present disclosure.

Referring to FIG. 15, the processor 1500 may include a motion information estimator 1510, a physical performance parameter extractor 1530 and a frailty index predictor 1550.

The motion information estimator 1510 may estimate motion information regarding the pose or shape of the subject, based on the distance information obtained by the distance sensor and the image information obtained by the image sensor.

As described above with reference to FIG. 14, the distance information obtained by the distance sensor may have an insufficient amount of data to extract motion information of a subject. For example, the distance information obtained by a ToF camera or a LiDAR sensor is characterized in that data is sparse. Accordingly, it may be very difficult to accurately estimate motion information such as the pose or shape of a subject using only the distance information obtained by the distance sensor. In contrast, the image information obtained by the image sensor does not include distance information, but is characterized in that data is dense compared to the distance information obtained by the distance sensor. Accordingly, in an embodiment, the processor 1500 may up-sample the distance information of the subject using the image information of the subject. For example, the processor 1500 may up-sample the distance information, by fusing the distance information and the image information based on the artificial neural network. In this case, in order to output high-resolution distance information by receiving low-resolution distance information and high-resolution image information as input, a super-resolution artificial neural network technique is applicable.

The motion information estimator 1510 may estimate the motion information of the subject using the artificial neural network. For example, the motion information estimator 1510 may estimate the motion information of the subject, by receiving the distance information and image information of the subject as input and executing a deep neural network (DNN).

The motion information estimator 1510 may estimate the motion information of the subject using the distance information in addition to the image information of the subject unlike the existing case, thereby more improving accuracy and reliability of an estimation result.

In addition, motion information including relative distance information may be estimated in the existing case, whereas the motion information estimator 1510 may estimate motion information including absolute distance information. Therefore, based on a parameter essentially requiring absolute distance information of a subject among various physical performance parameters, for example, a gait parameter, etc., a frailty index of the subject may be predicted. Specifically, the motion information estimator 1510 may detect a body key point of the subject from the distance information and image information of the subject using a deep neural network (DNN) model specialized for estimation of the pose and shape of the subject, and estimate absolute coordinates for the detected body key point. That is, existing 2D/3D pose estimators may output only relative coordinates of the subject, whereas the motion information estimator 1510 may output the absolute coordinates of the subject using the DNN model. Therefore, the embodiments of the present disclosure are applicable to physical performance parameters requiring motion information such as the gait speed of the subject.

Meanwhile, in an example, the artificial neural network for estimating the motion information may configure a network different from the artificial neural network for up-sampling distance information.

The physical performance parameter extractor 1530 may extract various physical performance parameters of the subject from the motion information obtained from the motion information estimator 1510. For example, the physical performance parameter extractor 1530 may extract the gait speed, balance time, sit-to-stand time, TUG (timed-up-and-go) time, etc. of the subject.

In order to extract the physical performance parameter of the subject, the physical performance parameter extractor 1530 may primarily recognize and classify a test situation. For example, the physical performance parameter extractor 1530 may recognize and classify whether a current test situation is a gait situation, sit-to-stand situation or balance situation of the subject. The test situation recognition and classification operation may be performed using user input or an action recognition artificial neural network. Here, the action recognition artificial neural network may refer to a network capable of identifying different actions within an input image (that is, motion information of the subject). In the case of the action recognition artificial neural network, after extracting spatial and temporal characteristics from the input image, a result value obtained by fusing the spatial and temporal characteristics may be output.

The physical performance parameter extractor 1530 may extract the physical performance parameter of the subject to be obtained in the current test situation from the motion information, after recognizing the test situation. The method of extracting the physical performance parameter from the motion information may be variously determined according to the test situation or the type of the parameter to be obtained. For example, referring to FIG. 16, in order to extract the gait parameter in the gait test situation of the subject, the physical performance parameter extractor 1530 may simulate the gait trajectory of the subject in real time from the left/right ankle key point spatial/temporal information in the motion information, and extract the gate speed, speed per minute, etc. of the subject based on the gait trajectory.

The frailty index predictor 1550 may predict the frailty index of the subject based on the physical performance parameters of the subject obtained from the physical performance parameter extractor 1530. In addition, the frailty index predictor 1550 may quantitatively predict the physiological age, sarcopenia, fall-risk, etc. of the subject in addition to the frailty index.

In an example, the frailty index predictor 1550 may predict the frailty index of the subject using a predetermined regression equation obtained through a clinical research. In another example, the frailty index predictor 1550 may predict the frailty index of the subject using a predetermined learning model obtained by performing machine learning based on clinical research result data.

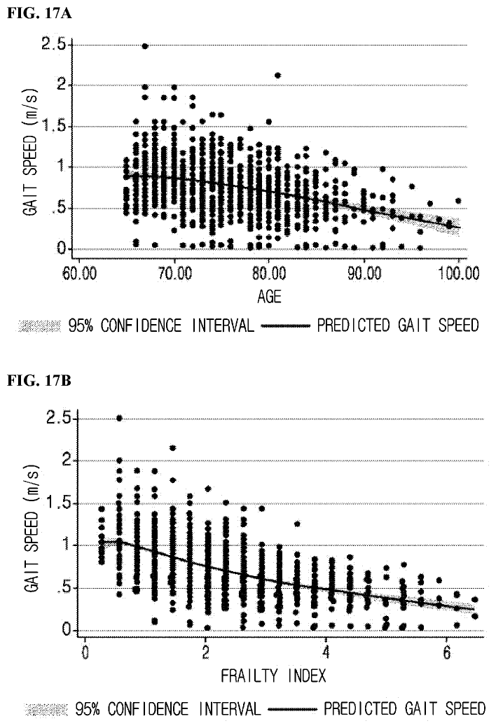

FIGS. 17a and 17b are two-dimensional graphs respectively illustrating a relationship between a chronological age and a gait speed and a relationship between a frailty index and a gait speed. The graphs of FIGS. 17a and 17b were obtained through linear regression analysis of statistical data, and the relationship between chronological age and gait speed and the relationship between frailty index and gait speed are shown in the form of a linear function in FIGS. 3A and 3B.

The x-axis in FIG. 17a refers to an average gait speed in m/s. In addition, the y-axis in FIG. 17a refers to actual age. Referring to FIG. 17a, it may be seen that the average gait speed decreases as the actual age increases. Therefore, it may be seen that the gait speed has a correlation with frailty.

The x-axis in FIG. 17b refers to an average gait speed in m/s. In addition, the y-axis in FIG. 17b refers to a frailty index. Referring to FIG. 17b, it may be seen that the average gait speed decreases as the frailty index increases. Therefore, it may be seen that the gait speed has a correlation with frailty.

As a result, referring to FIGS. 17a and 17b, the physiological age and frailty index of a frailty diagnosis-target person may be estimated by measuring the gait speed of the subject.

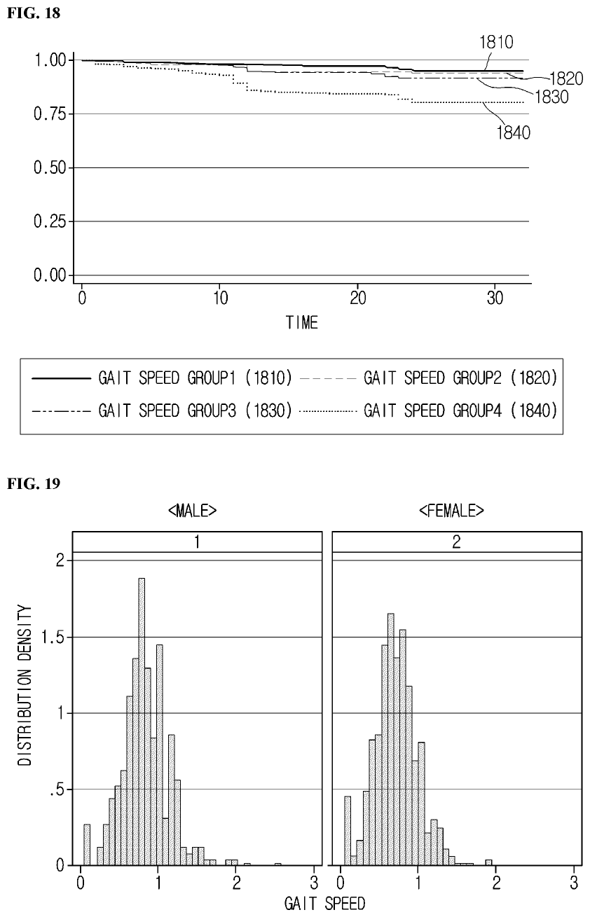

FIG. 18 is a graph illustrating the survival rates of groups having certain gait speeds by Kaplan-Meier analysis. Kaplan-Meier analysis concerns the probability of survival over time of people having specific conditions, which may be obtained by observing a sufficiently large sample population for a long period of time.

The x-axis in FIG. 18 refers to the proportion of survivors to the total cohort participants, and the y-axis in FIG. 18 refers to a measurement period Referring to FIG. 18, the cohort participants are classified into four groups 410, 420, 430, and 440 according to the gait speeds thereof, and results of observation of deaths over the measurement period are shown for each group. The proportion of survivors in the group 440 having the lowest gait speed has decreased the most, and the proportion of survivors in the group 410 having the highest gait speed has decreased the least. That is, the higher gait speed the group has, the higher survival rate the group has.

As a result, referring to FIG. 18, the survival rate of a subject may be predicted by measuring the gait speed of the subject.

Table 1 below shows items strongly related to the speed of walking. When a population group is divided into participants (high speed walkers) who walk faster than the median speed and participants (low speed walkers) who walk slower than the median speed, statistically significant differences are observed therebetween in multimorbidity, grip strength, short physical performance battery (SPPB), frailty indexes (K-FRAIL and CHS frailty score), activities of daily living and instrumental activities of daily living (ADL, IADL), depression, cognition, polypharmacy, fall history, etc. That is, it is possible to infer the health state of a frailty diagnosis-target person by measuring the gait speed of the frailty diagnosis-target person.

TABLE 1

| | Low speed | High speed | |
|---|---|---|---|
| Items | walkers | walkers | P value |
| multimorbidity (n) | 310.00 | 221.00 | <0.001 |
| Dominant grip strength | 19.92 | 24.90 | <0.001 |
| (mean, sd) | | | |
| SPPB score (mean, sd) | 6.61 | 9.37 | <0.001 |
| K-FRAIL score (mean, sd) | 1.63 | 0.93 | <0.001 |
| CHS score (mean, sd) | 2.39 | 1.25 | <0.001 |
| ADL disability (n, %) | 125.00 | 56.00 | <0.001 |
| IADL disability (n, %) | 294.00 | 150.00 | <0.001 |
| Depression (n, %) | 102.00 | 34.00 | <0.001 |
| Cognitive dysfunction (n, %) | 270.00 | 125.00 | <0.001 |
| Polypharmacy (n, %) | 193.00 | 113.00 | <0.001 |
| Fall history for previous 1 | 0.33 | 0.16 | 0.001 |
| year (mean, sd) | | | |

FIG. 19 illustrates distributions of the gait speed of older adults living in a community in Korea according to the genders. The left graph in FIG. 19 shows a distribution of the gait speed of men. The right graph in FIG. 19 shows a distribution of the gait speed of women. Since there is a difference in the distribution of gait speed between men and women, it is necessary to consider the gender of a frailty diagnosis-target person in addition to the gait speed of the frailty diagnosis-target person when measuring the physiological age of the frailty diagnosis-target person.

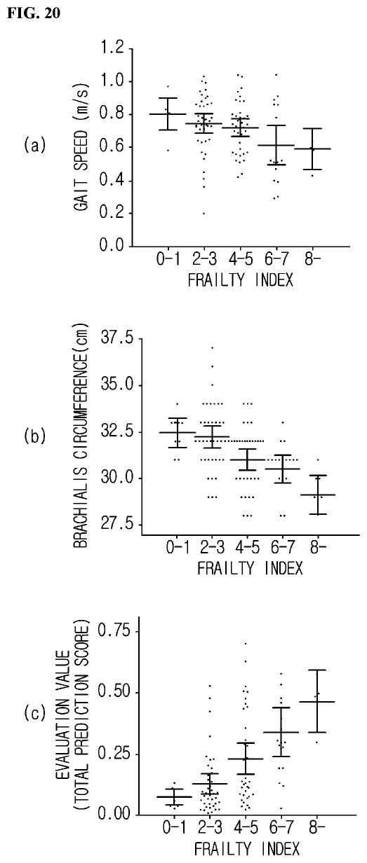

FIG. 20 is a graph for estimating a frailty index by using a gait speed and other measured values. Referring to FIG. 20, the speed of walking and the circumference of brachialis are measured to show a correlation between the frailty index and the sum of a gait speed parameter and a brachialis circumference parameter. The brachialis circumference parameter is related to the muscle function of a frailty diagnosis-target person and is thus closely related to the frailty index of the frailty diagnosis-target person. Thus, the brachialis circumference parameter is an important factor together with the speed of walking when estimating a frailty index.

The left graph of FIG. 20 shows a correlation between the gait speed parameter and the frailty index. Referring to the left graph, as the gait speed parameter decreases, the frailty index increases.

The middle graph of FIG. 20 shows a correlation between the brachialis circumference parameter and the frailty index. Referring to the middle graph, as the brachialis circumference parameter decreases, the frailty index increases.

The right graph of FIG. 20 shows a correlation between the frailty index and an evaluation value obtained based on the gait speed parameter and the brachialis circumference parameter according to the results shown in the left and middle graphs of FIG. 20. Referring to the right graph, as the evaluation value increases, the frailty index increases. The accuracy of estimation of a frailty index may increase by considering two or more factors in combination. In FIGS. 6A to 6C, the evaluation value obtained by combining the gait speed and the brachial circumference is used. However, another factor may be used instead of or in addition to the brachialis circumference to estimate the frailty index.

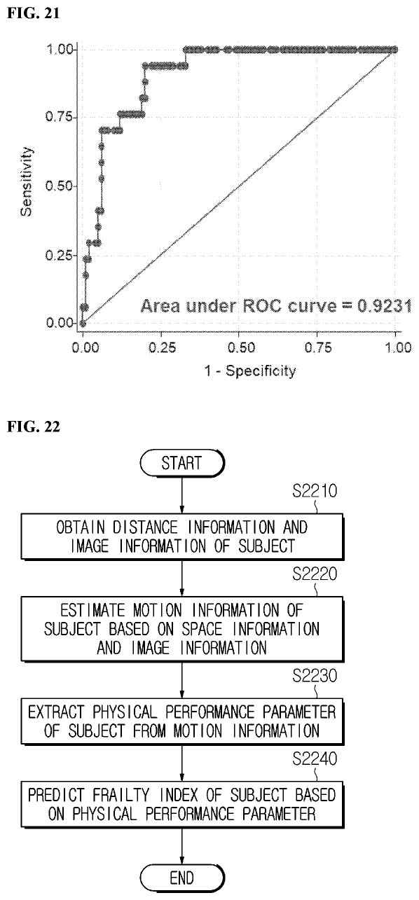

FIG. 21 shows frailty index prediction sensitivity and specificity by a physical function parameter. Referring to FIG. 21, it can be seen that the physical function parameter may be very effectively predicted through the fact that the width AUC of the lower region of the graph is greater than 0.9.

FIG. 22 is a flowchart illustrating a frailty index prediction method according to an embodiment of the present disclosure. The frailty index prediction method of FIG. 22 may be performed by the frailty index prediction device 1400 described above with reference to FIG. 14.

Referring to FIG. 22, the frailty index prediction device may obtain distance information and image information of a subject through the sensor (S2210). The sensor may include a single distance sensor and a single image sensor. The distance sensor outputs inspection waves of various waveforms, such as ultrasonic waves, infrared rays, lasers, etc. and detect the inspection waves reflected by the subject, thereby obtaining distance information regarding a distance and direction from an observation point to the subject. In an example, the distance sensor may include at least one of a ToF (Time of Flight) sensor, an ultrasonic sensor, a structured light sensor, an infrared sensor or a LiDAR sensor. The image sensor may obtain image information of the subject, by detecting a light signal output from a light source and reflected by the subject. In this specification, the image sensor may be referred to as a color camera. In an example, the image sensor may include a light source, a pixel array and a light detection circuit.

In an example, the sensor may have a single camera structure including a single distance sensor and a single image sensor. In addition, since the distance sensor and the image sensor may be integrally implemented as one module, a separate test space for sensor installation does not need to be secured and a compact form factor may be achieved.

The frailty index prediction device may estimate motion information regarding a pose or shape of the subject using the distance information and image information of the subject (S2220). For example, the frailty index prediction device may input the up-sampled distance information and image information of the subject to a DNN (Deep Neural Network) and estimate an output value obtained by executing the DNN as motion information of the subject. In an embodiment, the distance information of the subject may be up-sampled by inputting the distance information and the image information and executing the artificial neural network. In this case, a super-resolution artificial neural network technique for increasing resolution of the distance information is applicable to the artificial neural network.

The frailty index prediction device may extract various physical performance parameters of the subject from the motion information of the subject (S2230). The physical performance parameters may include a gait speed, a balance time, a sit-to-stand time, a TUG (timed-up-and-go) time, etc. The types of the physical performance parameters extracted from the motion information of the subject may vary according to a test situation recognized by user input or an action recognition artificial neural network. For example, in the gait test situation of the subject, the gait trajectory of the subject may be simulated in real time from the left/right ankle key point spatial/temporal information in the motion information, and the gait parameter such as the gate speed, speed per minute, etc. of the subject may be extracted based on the gait trajectory.

In an example, in order to extract the physical performance parameters of the subject, the motion information may be used.

The frailty index prediction device may predict a frailty index of the subject based on the physical performance parameters of the subject (S2240). In addition, the frailty index prediction device may predict the physiological age, sarcopenia, fall-risk, etc. of the subject in addition to the frailty index.

The above-described embodiments may be written as computer-executable programs and may be implemented in general-purpose digital computers that execute the programs using a non-transitory computer-readable recording medium.

The terms used in the present specification are selected based on general terms currently widely used in the art in consideration of functions regarding the present disclosure, but the terms may vary according to the intention of those of ordinary skill in the art, precedents, or new technology in the art. Also, some terms may be arbitrarily selected by the applicants, and in this case, the meaning of the selected terms are described in the detailed description of the present disclosure. Thus, the terms used herein should not be construed based on only the names of the terms but should be construed based on the meaning of the terms together with the description throughout the present disclosure.

The terms of a singular form may include plural forms unless referred to the contrary. In the present specification, when it is described that a part or portion “includes” and/or “comprises” a particular element, the presence or addition of one or more other elements in the part or portion is not precluded, and the part or portion may include or comprise one or more other elements unless otherwise specified.

While some best embodiments of the present disclosure have been described, it will be apparent to those of ordinary skill in the art that substitutions, modifications, and changes may be made therefrom. That is, the claims may include all of such substitutions, modifications, and changes. Therefore, all described in the present specification including the drawings should be construed in an illustrative and non-limiting sense.

### INDUSTRIAL APPLICABILITY

The embodiments according to the present disclosure may be used to predict a cardiopulmonary function index or frailty index of a subject.

1. A method of predicting a cardiopulmonary function, the method comprising:
measuring, by a motion sensor, motion of a cardiopulmonary function measurement-target person;
determining, by a processor, a gait speed of the cardiopulmonary function measurement-target person according to the motion of the cardiopulmonary function measurement-target person; and
predicting, by the processor, a cardiopulmonary function index of the cardiopulmonary function measurement-target person, based on the gait speed of the cardiopulmonary function measurement-target person.

2. The method of claim 1, wherein the predicting the cardiopulmonary function index comprises:
determining, by the processor, a gait speed parameter indicating the gait speed; and
predicting, by the processor, the cardiopulmonary function index according to the gait speed parameter.

3. The method of claim 2, wherein the gait speed parameter represents a gait speed of a specific section.

4. The method of claim 2, wherein the gait speed parameter is determined by a gait speed-cardiopulmonary function index correlation function indicating a relationship between the gait function and the cardiopulmonary function index.

5. The method of claim 1,
wherein the measuring the motion of the cardiopulmonary function measurement-target person comprises measuring two-dimensional motion of the cardiopulmonary function measurement-target person,
wherein the determining the gait speed of the cardiopulmonary function measurement-target person comprises measuring vertical and horizontal components of the gait speed, based on the two-dimensional motion of the cardiopulmonary function measurement-target person, and
wherein the predicting the cardiopulmonary function index comprises predicting the cardiopulmonary function index of the cardiopulmonary function measurement-target person, according to the vertical and horizontal components of the gait speed of the cardiopulmonary function measurement-target person.

6. The method of claim 1, wherein the predicting the cardiopulmonary function index comprises predicting the cardiopulmonary function index of the cardiopulmonary function measurement-target person, by further considering personal information of the cardiopulmonary function measurement-target person.

7. The method of claim 1,
wherein the determining the gait speed of the cardiopulmonary function measurement-target person comprises generating a time-based graph of the gait speed of the cardiopulmonary function measurement-target person, and
wherein the predicting the cardiopulmonary function index comprises predicting the cardiopulmonary function index of the cardiopulmonary function measurement-target person according to a gait pattern shown in the time-based graph of the gait speed.

8. The method of claim 1, further comprising measuring oxygen saturation of the cardiopulmonary function measurement-target person,
wherein the predicting the cardiopulmonary function index comprises predicting the cardiopulmonary function index of the cardiopulmonary function measurement-target person, by considering the oxygen saturation.

9. The method of claim 1, wherein the measuring the motion of the cardiopulmonary function measurement-target person comprises:
setting an effective measurement range of the motion sensor; and
measuring the motion of the cardiopulmonary function measurement-target person within the effective measurement range.

10. The method of claim 1, wherein the measuring the motion of the cardiopulmonary function measurement-target person comprises:
selecting one of a gait speed measurement mode in which the gait speed of the cardiopulmonary function measurement-target person is measured and a gait analysis mode in which the motion information according to gait analysis of the cardiopulmonary function measurement-target person is additionally obtained in addition to the gait speed; and
measuring the motion of the cardiopulmonary function measurement-target person according to the selected measurement mode.

11. The method of claim 1,
wherein the determining the gait speed of the cardiopulmonary function measurement-target person comprises determining gait acceleration of the cardiopulmonary function measurement-target person from the gait speed of the cardiopulmonary function measurement-target person, and
wherein the predicting the cardiopulmonary function index comprises the cardiopulmonary function index of the cardiopulmonary function measurement-target person, according to the gait speed and the gait acceleration.

12. A device for predicting a cardiopulmonary function, the device comprising:
a motion sensor configured to measure motion of a cardiopulmonary function measurement-target person;
a memory configured to store a program including at least one instruction; and
a processor configured to predict a cardiopulmonary function index of the cardiopulmonary function measurement-target person, by executing the at least one program,
wherein the at least one instruction comprises:
an instruction for enabling the motion sensor to measure motion of the cardiopulmonary function measurement-target person;
an instruction for enabling the processor to determine at least one of a gait speed or gait acceleration of the cardiopulmonary function measurement-target person according to the motion of the cardiopulmonary function measurement-target person; and
an instruction for enabling the processor to predict a cardiopulmonary function index of the cardiopulmonary function measurement-target person, based on the gait speed or gait acceleration of the cardiopulmonary function measurement-target person.

13. The device of claim 12, wherein the motion sensor is a 2D Lidar.

14. A computer program product comprising instructions for causing each step of the method of predicting the cardiopulmonary function of claim 1 to be performed by a computer.

15. A computer-readable recording medium storing the computer program of claim 14.

16. A motion-based frailty index prediction method performed by a frailty index prediction device comprising a sensor and a processor, the motion-based frailty index prediction method comprising:
obtaining, by the sensor, distance information and image information of a subject;
estimating, by the processor, motion information of the subject using the distance information and the image information;
extracting, by the processor, a physical function parameter of the subject from the motion information; and
predicting, by the processor, a frailty index of the subject based on the physical function parameter,
wherein the sensor comprises a single distance sensor for obtaining the distance information and a single image sensor for obtaining the image information.

17. The motion-based frailty index prediction method of claim 16, wherein the distance information is up-sampled using the image information.

18. The motion-based frailty index prediction method of claim 17, wherein the distance information is up-sampled by receiving the distance information and the image information as input and executing an artificial neural network.

19. The motion-based frailty index prediction method of claim 18, wherein a super-resolution artificial neural network technique is applied to the artificial neural network.

20. The motion-based frailty index prediction method of claim 16, wherein the motion information comprises absolute distance information regarding a body of the subject.

21. The motion-based frailty index prediction method of claim 16, wherein the estimating the motion information is performed by receiving the distance information and the image information as input and executing a deep neural network (DNN).

22. The motion-based frailty index prediction method of claim 16, wherein the physical function parameter comprises at least one of a gait speed, balance time, sit-to-stand time or TUG (timed-up-and-go) of the subject.

23. The motion-based frailty index prediction method of claim 16, wherein the extracting the physical function parameter comprising:
classifying operation situations of the subject; and
extracting the physical function parameter from the motion information, based on the classified operation situations of the subject.

24. The motion-based frailty index prediction method of claim 23, wherein the classifying the operation situations of the subject is performed based on user input or an action recognition artificial neural network.

25. The motion-based frailty index prediction method of claim 16, wherein the single distance sensor comprises a ToF (Time of Flight) sensor or a LiDAR sensor.

26. The motion-based frailty index prediction method of claim 16, wherein the motion information comprises at least one of a pose or shape of the subject.

27. The motion-based frailty index prediction method of claim 16, wherein a result derived in the predicting the frailty index comprises at least one of a frailty index, a physiological age, sarcopenia or a fall-risk.

28. The motion-based frailty index prediction method of claim 16, wherein the physical function parameter is extracted from the motion information using the motion information.

29. A computer program product comprising instructions performed by a computer for a motion-based frailty index prediction method,
wherein the motion-based frailty index prediction method is performed by a frailty index prediction device comprising a sensor and a processor,
wherein the motion-based frailty index prediction method comprising:
obtaining, by the sensor, distance information and image information of a subject;
estimating, by the processor, motion information of the subject using the distance information and the image information;
extracting, by the processor, a physical function parameter of the subject from the motion information; and
predicting, by the processor, a frailty index of the subject based on the physical function parameter,
wherein the sensor comprises a single distance sensor for obtaining the distance information and a single image sensor for obtaining the image information.

30. A motion-based frailty index prediction device comprising:
a single distance sensor configured to detect distance information of a subject;
a single image sensor configured to detect image information of the subject; and
a processor configured to predict a frailty index of the subject,
wherein the processor is configured to:
estimate motion information of the subject using the distance information and the image information,
extract a physical function parameter of the subject from the motion information, and predict the frailty index of the subject based on the extracted physical function parameter.

31. The motion-based frailty index prediction device of claim 30, wherein the distance information is up-sampled using the image information.

32. The motion-based frailty index prediction device of claim 31, wherein the distance information is up-sampled by receiving the distance information and the image information as input and executing an artificial neural network.

33. The motion-based frailty index prediction device of claim 32, wherein a super-resolution artificial neural network technique is applied to the artificial neural network.

34. The motion-based frailty index prediction device of claim 30, wherein the motion information comprises absolute distance information regarding a body of the subject.

35. The motion-based frailty index prediction device of claim 30, wherein the processor estimates the motion information by receiving the distance information and the image information as input and executing a deep neural network (DNN).

36. The motion-based frailty index prediction device of claim 30, wherein the physical function parameter comprises at least one of a gait speed, balance time, sit-to-stand time or TUG (timed-up-and-go) of the subject.

37. The motion-based frailty index prediction device of claim 30, wherein operation situations of the subject are classified and the physical function parameter is estimated from the motion information, based on the classified operation situations of the subject.

38. The motion-based frailty index prediction device of claim 37, wherein the operation situations of the subject are classified based on user input or an action recognition artificial neural network.

39. The motion-based frailty index prediction device of claim 30, wherein the single distance sensor comprises a ToF (Time of Flight) sensor or a LiDAR sensor.

40. The motion-based frailty index prediction device of claim 30, wherein the motion information comprises at least one of a pose or shape of the subject.

41. The motion-based frailty index prediction device of claim 30, wherein the processor predicts at least one of a frailty index, a physiological age, sarcopenia or a fall-risk of the subject, based on the extracted physical function parameter.

42. The motion-based frailty index prediction device of claim 30, wherein the processor extracts the physical function parameter using the motion information.
